(19) United States
(12) Patent Application Publication
(10) Pub. No.: US 2004/0005566 A1
(43) Pub. Date: Jan. 8, 2004

(54) Kits and methods for assessing cardiovascular health

(75) Inventors: John R. DePhillipo, Margate, NJ (US); Robert P. Ricciardi, Glen Mills, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Genelink, Inc., Margate, NJ (US)

(21) Appl. No.: 10/188,701

(22) Filed: Jul. 2, 2002

(51) Int. Cl. C12Q 1/68

(52) U.S. Cl. 435/6

(57) ABSTRACT

The invention relates to kits and methods for assessing the cardiovascular health of a human and the human's susceptibility to cardiovascular disorders. The methods involve assessing occurrence in the human's genome of one or more polymorphisms (e.g., single nucleotide polymorphisms) that occur in one or more genes associated disclosed herein and that are associated with a disorder in humans. Preferred assessment and scoring methods are disclosed, as are kits for performing the methods.

### BACKGROUND OF THE INVENTION

Blood transports oxygen, glucose, lipids, vitamins, hormones, immune system components, and other materials to substantially all parts of the body and removes wastes therefrom. Blood is contained and moved by tissues of the cardiovascular system, including the heart and blood vessels such as arteries, veins, and capillaries. A properly functioning cardiovascular system is critical to human health.

Cardiovascular disorders are common in modem populations, and are the cause of significant morbidity and mortality. Among the more common cardiovascular disorders are coronary artery diseases (CADs), congenital heart defects, cardiac arrhythmias, congestive heart failure (CHF), and vascular disorders.

CADs, sometimes designated coronary heart diseases or ischemic heart diseases, are characterized by insufficiency in blood supply to cardiac muscle. CADs can be manifested as acute cardiac ischemia (e.g., angina pectoris or myocardial infarction) or chronic cardiac ischemia (e.g., coronary arteriosclerosis or coronary atherosclerosis). CADs are a common cause of disability, including cardiac failure and cardiac arrhythmias, and sudden death. In patients afflicted with CADs, the cardiac muscle is not sufficiently supplied with oxygen. Severe cardiac ischemia can be manifested as severe pain or cardiac damage. Less severe ischemia can damage cardiac muscle and cause changes to cardiac tissues over the long term that impair cardiac function.

The etiology of many CADs is not thoroughly understood. It is understood that many CADs are interrelated. For example, arteriosclerosis, atherosclerosis, or vasospasm in coronary arteries can lead to development of angina pectoris. Also, some environmental or dietary factors (e.g., chronic high fat intake) are known to predispose individuals to CADs Nonetheless, the physiological, environmental, or genetic factors that influence the likelihood that an individual will develop a CAD are not well known.

Vascular disorders include arteriosclerosis, atherosclerosis, hypertension, aneurysm, phlebothrombosis, several less common disorders (e.g., Buerger's disease and Raynaud's syndrome), and varicose veins. As with CADs, the etiologies of many vascular disorders are not well understood, although disposing factors are often known. For example, a high fat diet is know to raise the likelihood that a patient will develop arteriosclerosis or atherosclerosis. High sodium content in the diet is commonly believed to predispose one to hypertension. However, dietary effects do not appear to have the same effect on all individuals. Some individuals do not develop arteriosclerosis or hypertension despite consuming diets high in fat and sodium. Genetic factors are believed to contribute to an individual's susceptibility to vascular disorders, but these genetic factors have not been well characterized previously.

Several types of hypertension (“high blood pressure”) are known. Some have known etiologies, such as secondary hypertension that results from renal or endocrine dysfunction. The etiologies of other forms of hypertension (e.g., primary and malignant hypertension) are less well understood, and genetic factors likely contribute more heavily than they do to etiology of secondary hypertension. Blood pressure is modulated in part by the contraction and resilience of blood vessels. Many factors, including genetic factors, influence a patient's blood pressure and the patient's blood pressure response to environmental, dietary, and other stimuli.

Properties of a patient's blood vessels (including the patient's genetic composition) influence the normal and diseased condition of the vessels and their interaction environmental compounds and with other body components. Arteriosclerosis refers to changes that occur in blood vessel wall, regardless of the reason for the change. Arteriosclerosis is more common in diabetic patients and as patients age. Nonetheless, rates of arteriosclerosis vary among individuals. Atherosclerosis is characterized by occurrence of atheromas in or on vascular walls. Atheromas are plaques that can contain lipids, cells, extracellular matrix components, and other materials. Thrombi commonly occur on the lumenal face of atherosclerotic blood vessels. Atherosclerotic thrombi can narrow blood vessels (e.g., contributing to hypertension) and can detach and obstruct blood vessels in other parts of the body. Detached atherosclerotic thrombi can lead to stroke or myocardial infarction if the thrombi lodge in blood vessels supplying cerebral or cardiac tissues. In some patients, thrombi spontaneously form in blood vessels, particularly commonly in deep veins of the leg. In some patients, thrombus formation occurs primarily in inflamed blood vessels; the disorder is often referred to as thrombophlebitis in these patients. In other patients, veins in which thrombi form are not necessarily inflamed; the disorder is often referred to as phlebothrombosis (or “deep vein thrombosis”) in these patients.

The etiologies of cardiac arrhythmias and congestive heart failure are more complicated. Sometimes, these conditions do not appear to be related to any other cardiovascular disorder. Often, however, arrhythmias and congestive heart failure (or cardiac insufficiency) develop as a result of other long-standing cardiovascular disorders, such as the CADs and vascular disorders described above.

Most, if not all, human genes occur in a variety of forms which differ in at least minor ways. Heterogeneity in human genes is believed to have arisen, in part, from minor, non-fatal mutations that have occurred in the genome over time. In some instances, differences between alternative forms of a gene are manifested as differences in the amino acid sequence of a protein encoded by the gene. Some amino acid sequence differences can alter the reactivity or substrate specificity of the protein. Differences between alternative forms of a gene can also affect the degree to which (if at all) the gene is expressed. However, many heterogeneities that occur in human genes appear not to be correlated with any particular phenotype. Known heterogeneities include, for example, single nucleotide polymorphisms (i.e., alternative forms of a gene having a difference at a single nucleotide residue). Other known polymorphic forms include those in which the sequence of larger (e.g., 2-1000 residues) portions of a gene exhibits numerous sequence differences and those which differ by the presence or absence of portion of a gene.

Numerous disorders and physiological states have been correlated with occurrence of one or more alternative forms of an individual gene in the genome of a human who exhibits the disorder or physiological state. For example, Kimura et al. (2000, Am. J. Ophthalmol. 130:769-773) discloses an association between occurrence of a SNP of the manganese superoxide dismutase gene and a form of macular degeneration.

Associations between individual disorders and individual genetic polymorphisms are known. However, disorders can usually result from polymorphisms in any of a relatively large number of genes, and as a result, assessing the polymorphic form(s) of any single gene that occur in a human's genome is usually not predictive of the overall likelihood that the human will develop the disorder.

Many disorders, including many cardiovascular disorders, develop over time and could be delayed, inhibited, lessened in severity, or prevented altogether by making lifestyle changes. For cardiovascular disorders, such changes include increasing exercise, adjusting diet, consuming nutritional or pharmaceutical products known to be effective against cardiovascular disorders, and undergoing heightened medical monitoring. These changes are often not made, owing to, the expense or inconvenience of the changes and one's subjective belief that one is not at high risk for cardiovascular disorders. Improved monitoring of cardiovascular health can help to identify individuals at risk for developing cardiovascular disorders and permit more informed decisions to be made regarding whether lifestyle changes are justified.

A need remains for a method of assessing the overall state of a person's cardiovascular health, or a human's predisposition to develop one or more cardiovascular disorders. The invention satisfies this need.

### BRIEF SUMMARY OF THE INVENTION

The invention relates to a method of assessing cardiovascular health in a human. The method comprises assessing occurrence in the human's genome of disorder-associated polymorphisms in at least two (four, six, eight, ten, fifteen, or even all) genes selected from the group consisting of

a) the gene which encodes apolipoprotein B;

b) the gene which encodes apolipoprotein E;

c) the gene which encodes paraoxonase 1;

d) the gene which encodes type 1 angiotensin II receptor;

e) the gene which encodes cytochrome b-245(alpha);

f) the gene which encodes prothrombin;

g) the gene which encodes coagulation factor VII;

h) the gene which encodes platelet glycoprotein 1b alpha;

i) the gene which encodes platelet glycoprotein IIIa;

j) the gene which encodes endothelial nitric oxide synthase;

k) the gene which encodes 5,10-methylene tetrahydrofolate reductase;

l) the gene which encodes angiotensinogen;

m) the gene which encodes plasminogen activator inhibitor 1;

n) the gene which encodes coagulation factor V;

o) the gene which encodes alpha adducin I;

p) the gene which encodes cytochrome P450;

q) the gene which encodes G protein beta, polypeptide 3;

r) the gene which encodes methionine synthase reductase;

s) the gene which encodes endothelial adhesion molecule 1; and

t) the gene which encodes cholesteryl ester transferase.

Occurrence of any of the polymorphisms is an indication that the human has poorer cardiovascular health than a human whose genome does not comprise the polymorphism, and whereby occurrence of a plurality of the polymorphisms is an indication that the human has even poorer cardiovascular health than a human whose genome does not comprise the polymorphisms.

For example, occurrence of at least two disorder-associated polymorphisms can be assessed, where the polymorphisms are selected from the group consisting of

A) a polymorphism in the open reading frame encoding apolipoprotein B;

B) a polymorphism in the open reading frame encoding apolipoprotein E;

C) a polymorphism in the open reading frame encoding paraoxonase 1;

D) a polymorphism in the 3′-untranslated region of the gene encoding type 1 angiotensin II receptor;

E) a polymorphism in the open reading frame encoding cytochrome b-245(alpha);

F) a polymorphism in the 3′-untranslated region of the gene encoding prothrombin;

G) a polymorphism in the open reading frame encoding coagulation factor VII;

H) a polymorphism in the open reading frame encoding platelet glycoprotein 1b alpha;

I) a polymorphism in the open reading frame encoding platelet glycoprotein IIa;

J) a polymorphism in the open reading frame encoding endothelial nitric oxide synthase;

K) a polymorphism in the open reading frame encoding 5, 10-methylene tetrahydrofolate reductase;

L) a polymorphism in the open reading frame encoding angiotensinogen;

M) a polymorphism in the promoter operably linked with the open reading frame encoding plasminogen activator inhibitor 1;

N) a polymorphism in the open reading frame encoding coagulation factor V;

O) a polymorphism in the open reading frame encoding alpha adducin I;

P) a polymorphism in the promoter operably linked with the open reading frame encoding cytochrome P450;

Q) a polymorphism in the open reading frame encoding G protein beta, polypeptide 3;

R) a polymorphism in the open reading frame encoding methionine synthase reductase;

S) a polymorphism in the open reading frame encoding endothelial adhesion molecule 1; and

T) a polymorphism in the promoter operably linked with the open reading frame encoding cholesteryl ester transferase.

The polymorphism can be selected from the group consisting of

i) a polymorphism manifested as occurrence of a codon encoding glutamine at the codon including nucleotide residue 4154 of the open reading frame encoding apolipoprotein B;

ii) a polymorphism manifested as occurrence of a codon encoding lysine at the codon including nucleotide residue 4154 of the open reading frame encoding apolipoprotein B;

iii) a polymorphism manifested as occurrence of a codon encoding arginine at the codon including nucleotide residue 3611 of the open reading frame encoding apolipoprotein B;

iv) a polymorphism manifested as occurrence of a codon encoding glutamate at the codon including nucleotide residue 3611 of the open reading frame encoding apolipoprotein B;

v) a polymorphism manifested as occurrence of a codon encoding arginine at codon 112 of the open reading frame encoding apolipoprotein E;

vi) a polymorphism manifested as occurrence of a codon encoding cysteine at codon 112 of the open reading frame encoding apolipoprotein E;

vii) a polymorphism manifested as occurrence of a codon encoding arginine at codon 158 of the open reading frame encoding apolipoprotein E;

viii) a polymorphism manifested as occurrence of a codon encoding cysteine at codon 158 of the open reading frame encoding apolipoprotein E;

ix) a polymorphism manifested as occurrence of a codon encoding arginine at codon 192 of the open reading frame encoding apolipoprotein E;

x) a polymorphism manifested as occurrence of a codon encoding glutamine at codon 192 of the open reading frame encoding apolipoprotein E;

xi) a polymorphism manifested as occurrence of an adenosine residue at nucleotide residue 1166 in the 3′-untranslated region of the gene encoding type 1 angiotensin II receptor;

xii) a polymorphism manifested as occurrence of a cytosine residue at nucleotide residue 1166 in the 3′-untranslated region of the gene encoding type 1 angiotensin II receptor;

xiii) a polymorphism manifested as occurrence of a codon encoding histidine at codon 72 of the open reading frame encoding cytochrome b-245(alpha);

xiv) a polymorphism manifested as occurrence of a codon encoding tyrosine at codon 72 of the open reading frame encoding cytochrome b-245(alpha);

xv) a polymorphism manifested as occurrence of an adenosine residue at nucleotide residue 20210 in the 3′-untranslated region of the gene encoding prothrombin;

xvi) a polymorphism manifested as occurrence of a guanine residue at nucleotide residue 20210 in the 3′-untranslated region of the gene encoding prothrombin;

xvii) a polymorphism manifested as occurrence of a codon encoding arginine at codon 353 of the open reading frame encoding coagulation factor VII;

xviii) a polymorphism manifested as occurrence of a codon encoding glutamine at codon 353 of the open reading frame encoding coagulation factor VII;

xix) a polymorphism manifested as occurrence of a codon encoding threonine at codon 145 of the open reading frame encoding platelet glycoprotein 1b alpha;

xx) a polymorphism manifested as occurrence of a codon encoding methionine at codon 145 of the open reading frame encoding platelet glycoprotein 1b alpha;

xxi) a polymorphism manifested as occurrence of a codon encoding leucine at codon 33 of the open reading frame encoding platelet glycoprotein IIIa;

xxii) a polymorphism manifested as occurrence of a codon encoding proline at codon 33 of the open reading frame encoding platelet glycoprotein IIIa;

xxiii) a polymorphism manifested as occurrence of a codon encoding glutamate at codon 298 of the open reading frame encoding endothelial nitric oxide synthase;

xxiv) a polymorphism manifested as occurrence of a codon encoding aspartate at codon 298 of the open reading frame encoding endothelial nitric oxide synthase;

xxv) a polymorphism manifested as occurrence of a codon encoding alanine at codon 222 of the open reading frame encoding 5, 10-methylene tetrahydrofolate reductase;

xxvi) a polymorphism manifested as occurrence of a codon encoding valine at codon 222 of the open reading frame encoding 5, 10-methylene tetrahydrofolate reductase;

xxvii) a polymorphism manifested as occurrence of a codon encoding methionine at codon 235 of the open reading frame encoding angiotensinogen;

xxviii) a polymorphism manifested as occurrence of a codon encoding threonine at codon 235 of the open reading frame encoding angiotensinogen;

xxix) a polymorphism manifested as occurrence of a codon encoding threonine at codon 174 of the open reading frame encoding angiotensinogen;

xxx) a polymorphism manifested as occurrence of a codon encoding methionine at codon 174 of the open reading frame encoding angiotensinogen;

xxxi) a polymorphism manifested as occurrence of a tetra-guanine repeat near nucleotide residue −675 of the promoter operably linked with the open reading frame encoding plasminogen activator inhibitor 1;

xxxii) a polymorphism manifested as occurrence of a penta-guanine repeat near nucleotide residue −675 of the promoter operably linked with the open reading frame encoding plasminogen activator inhibitor 1;

xxxiii) a polymorphism manifested as occurrence of a codon encoding arginine at codon 506 of the open reading frame encoding coagulation factor V;

xxxiv) a polymorphism manifested as occurrence of a codon encoding glutamine at codon 506 of the open reading frame encoding coagulation factor V;

xxxv) a polymorphism manifested as occurrence of a codon encoding glycine at codon 460 of the open reading frame encoding alpha adducin I;

xxxvi) a polymorphism manifested as occurrence of a codon encoding tryptophan at codon 460 of the open reading frame encoding alpha adducin I;

xxxvii) a polymorphism manifested as occurrence of a cytosine residue at nucleotide residue −344 of the promoter operably linked with the open reading frame encoding cytochrome P450;

xxxviii) a polymorphism manifested as occurrence of a thymine residue at nucleotide residue −344 of the promoter operably linked with the open reading frame encoding cytochrome P450;

xxxix) a polymorphism manifested as occurrence of a cytosine residue at nucleotide residue 825 of the open reading frame encoding G protein beta, polypeptide 3;

xxxx) a polymorphism manifested as occurrence of a thymine residue at nucleotide residue 825 of the open reading frame encoding G protein beta, polypeptide 3;

xxxxi) a polymorphism manifested as occurrence of a codon encoding isoleucine at codon 22 of the open reading frame encoding methionine synthase reductase;

xxxxii) a polymorphism manifested as occurrence of a codon encoding methionine at codon 22 of the open reading frame encoding methionine synthase reductase;

xxxxiii) a polymorphism manifested as occurrence of a codon encoding serine at codon 128 of the open reading frame encoding endothelial adhesion molecule 1;

xxxxiv) a polymorphism manifested as occurrence of a codon encoding arginine at codon 128 of the open reading frame encoding endothelial adhesion molecule 1;

xxxxv) a polymorphism manifested as occurrence of a cytosine residue at nucleotide residue −629 of the promoter operably linked with the open reading frame encoding cholesteryl ester transferase;

xxxxvi) a polymorphism manifested as occurrence of an adenosine residue at nucleotide residue −629 of the promoter operably linked with the open reading frame encoding cholesteryl ester transferase;

xxxxvii) a polymorphism manifested as occurrence of a guanine residue at nucleotide residue −971 of the promoter operably linked with the open reading frame encoding cholesteryl ester transferase; and

xxxxviii) a polymorphism manifested as occurrence of an adenosine residue at nucleotide residue −971 of the promoter operably linked with the open reading frame encoding cholesteryl ester transferase.

Occurrence of an individual disorder-associated polymorphism can be assessed by first contacting a nucleic acid derived from the human's genome with a first oligonucleotide that anneals with higher stringency with the disorder-associated polymorphism than with a corresponding non-disorder-associated polymorphism and then assessing annealing of the first oligonucleotide and the nucleic acid. Annealing of the first oligonucleotide and the nucleic acid is an indication that the human's genome comprises the disorder-associated polymorphism.

Occurrence of an individual disorder-associated polymorphism can be further assessed by contacting the nucleic acid with a second oligonucleotide that anneals with higher stringency with a non-disorder-associated polymorphism than with the corresponding non-disorder-associated polymorphism and assessing annealing of the second oligonucleotide and the nucleic acid. Annealing of the second oligonucleotide and the nucleic acid is an indication that the human's genome does not comprise the disorder-associated polymorphism.

A cardiovascular health score can be calculated by summing, for each of the selected genes in which a disorder-associated polymorphism occurs in the human's genome, the product of a constant and a correlation factor. The correlation factor can, for example, represent the fraction of humans heterozygous or homozygous for the disorder-associated polymorphism who exhibit the corresponding disorder. The cardiovascular health score represents the relative susceptibility of the human to a cardiovascular disorder.

The same methods can be used to assess the likelihood that a human will develop a cardiovascular disorder. Occurrence of any of the polymorphisms is an indication that the human is more susceptible to the cardiovascular disorder than a human whose genome does not comprise the polymorphism, and occurrence of a plurality of the polymorphisms is an indication that the human is even more susceptible to the cardiovascular disorder than a human whose genome does not comprise the polymorphisms.

These methods can also be used to select a dose of a cardiovascular protective composition for administration to a human. Occurrence of any of the polymorphisms is an indication that a greater dose of the composition should be administered to the human. The dose of the composition can be selected based on occurrence of the polymorphisms.

The invention further relates to a kit for assessing relative susceptibility of a human to a cardiovascular disorder. The kit comprises reagents for assessing occurrence in the human's genome of disorder-associated polymorphisms in at least two of genes disclosed herein.

In another aspect, the invention relates to a method of assessing the advisability that a human should employ a nutritional product comprising a cardiovascular protective agent. The method comprises assessing occurrence in the human's genome of disorder-associated polymorphisms in at least two of the genes disclosed herein. Occurrence of any of the polymorphisms is an indication that it is more advisable for the human to employ the nutritional product than a human whose genome does not comprise the polymorphism, and occurrence of a plurality of the polymorphisms is an indication that it is even more advisable that the human should employ the nutritional product than a human whose genome does not comprise the polymorphisms.

In still another aspect, the invention relates to a method of selecting a dose of a cardiovascular protective agent for administration to a human in a nutritional product. The method comprises assessing occurrence in the human's genome of disorder-associated polymorphisms in at least two genes disclosed. Occurrence of any of the polymorphisms is an indication that a greater dose of the agent should be administered to the human in the nutritional product. The dose of the agent for the nutritional product can be selected based on occurrence of the polymorphisms.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. The invention is not limited to the precise arrangements and instrumentalities shown.

FIGS. 1A and 1B are images which depict examples of results that can be obtained by analyzing occurrence of polymorphisms in several genes. The results shown in FIG. 1A are derived from a hypothetical first human, and those shown in FIG. 1B are derived from a hypothetical second human. Circles represent different polymorphisms of the gene indicated to the left of the row of circles. Filled circles indicate the presence of the polymorphism. Non-filled circles indicate the absence of the polymorphism. Numbers below each circle represent a correlation factor for the polymorphism and a disease or disorder.

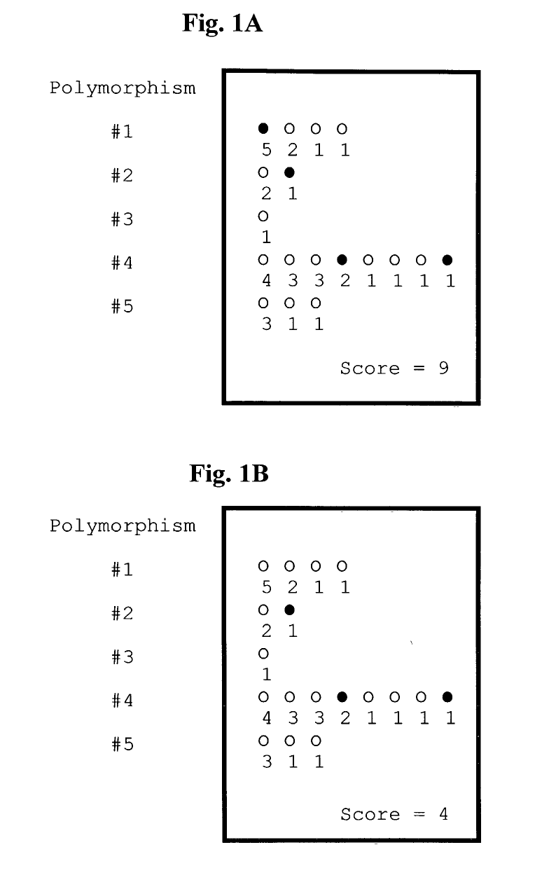

### DETAILED DESCRIPTION OF THE INVENTION

The invention relates to kits and methods for assessing cardiovascular health in a human by assessing occurrence in the human's genome of genetic polymorphisms that are associated with disorders. To better characterize the human's genetic content, occurrence of polymorphisms that are not associated with a disorder can also be assessed, so that one can determine whether the human is 1) homozygous for the disorder-associated polymorphism as a genomic site, 2) heterozygous for disorder-associated and disorder-non-associated polymorphisms at that site, or 3) homozygous for disorder-non-associated polymorphisms at that site. Assessments of genomic polymorphism content in two or more (and preferably in four, six, eight, ten, fifteen, or twenty) of the genes identified herein as being significant to cardiovascular health can be combined to indicate the cardiovascular health of the human. This assessment of cardiovascular health can be used to predict the likelihood that the human will develop (i.e., is predisposed to develop) a cardiovascular disorder, such as a CAD (e.g., myocardial infarction), hypertension, or phlebothrombosis.

Crudely simplified, the methods involves determining whether two or more polymorphisms that have been associated (by the inventors or by others) with a disorder (e.g., a disease or pathological state, whether of the cardiovascular system or not) in humans occur in the genome of the human being tested. In some embodiments, the number of polymorphisms that occur in the human's genome are summed to yield a value; the higher the value is, the greater the susceptibility of the human to cardiovascular disorders is assessed to be (i.e., the lower the human's cardiovascular health is assessed to be). In other embodiments, a weighting factor is assigned to each polymorphism tested, and the weighting factors of polymorphisms that occur in the human's genome are summed to yield a value that represents relative cardiovascular health (e.g., as assessed by susceptibility to cardiovascular disorders). The weighting factor can represent the product of a constant assigned to the gene in which the corresponding polymorphism occurs and a correlation factor that describes how informative occurrence of the polymorphism is for occurrence of the disorder with which it is associated. The weighting factor can also be influenced by whether the human is homozygous or heterozygous for the disorder-associated polymorphism. The invention includes a variety of alternative methods and kits for performing the methods, as described in greater detail herein.

Definitions

As used in this disclosure, the following terms have the meanings associated with them in this section.

A “polymorphism” in a gene is one of the alternative forms of a portion of the gene that are known to occur in the human population. For example, many genes are known to exhibit single nucleotide polymorphic forms, whereby the identity of a single nucleotide residue of the gene differs among the forms. Each of the polymorphic forms represents a single polymorphism, as the term is used herein. Other known polymorphic forms include alternative forms in which multiple consecutive or closely-spaced, non-consecutive nucleotide residues vary in sequence, forms which differ by the presence or absence of a single nucleotide residue or a small number of nucleotide residues, and forms which exhibit different mRNA splicing patterns.

A “single nucleotide polymorphism” (“SNP”) is one of the alternative forms of a portion of a gene that vary only in the identity of a single nucleotide residue in that portion.

A “disorder-associated” polymorphism is an alternative form of a portion of a gene, wherein occurrence of the alternative form in the genome of a human has been correlated with exhibition by the human of a disease or a pathological state.

A “disorder-non-associated” polymorphism is an alternative form of a portion of a gene for which no significant correlation has been made between occurrence of the alternative form in the genome and a disease or a pathological state. Disorder-non-associated polymorphisms are sometimes designated “neutral” polymorphisms in the art.

A disorder-associated polymorphism and a disorder-non-associated polymorphism “correspond” with one another if the two polymorphisms are two alternative forms of the same portion of the gene. By way of example, if the identity of residue 100 of a gene is adenine in a disorder-associated polymorphism of the gene and cytosine in a disorder-non-associated polymorphism of the gene, then the two polymorphisms correspond with one another. It is understood that there may be three or more corresponding polymorphisms when there are more than two alternative forms of the same portion of the gene.

A “characteristic residue” of a polymorphism is a nucleotide residue, the identity of which is known to vary among the alternative forms corresponding to the polymorphism.

“Cardiovascular health” means the likelihood that a human is experiencing, or is likely to experience in the future, a cardiovascular disorder.

A “cardiovascular disorder” is a pathological condition characterized by dysfunction of some portion of the circulatory system, such as dysfunctions of the heart, coronary blood vessels, and peripheral blood vessels (e.g., arteries, veins, and capillaries).

A “coronary artery disease” (“CAD”) is a pathological state characterized by insufficiency of oxygen delivery to cardiac muscle, wherein the condition is associated with some dysfunction of coronary blood vessels. As used in this disclosure, CADs include both disorders in which symptomatic and/or asymptomatic cardiac ischemia occurs (e.g., angina pectoris and myocardial infarction) and disorders that gradually lead to chronic or acute cardiac ischemia, even at the stage of the disorder at which such ischemia is not yet evident (e.g., coronary arteriosclerosis and atherosclerosis).

A “molecular beacon oligonucleotide” is a single-stranded oligonucleotides having a fluorescent label (e.g., rhodamine, FAM, TET, VIC, JOE, or HEX) attached to the 5′-end thereof and a fluorescence quencher (e.g., TAMRA or DABCYL) attached to the 3′-end thereof (or vice versa), as described (Kostrikis et al., 1998, Science 279:1228-1229).

Two molecular beacon oligonucleotides are “spectrally distinct” if they can be differentially detected using spectrophotometric or spectrofluorimetric methods. Examples of characteristics that can be used to differentiate spectrally distinct oligonucleotides include absorption or excitation wavelength, emission wavelength, and fluorescent lifetime.

An “instructional material” is a publication, a recording, a diagram, or any other medium of expression which can be used to communicate how to use a kit described herein, numerical values for weighting the significance of various polymorphisms that are detectable using the kit, or both. The instructional material of the kit of the invention can, for example, be affixed to a container which contains a kit of the invention or be shipped together with a container which contains the kit. Alternatively, the instructional material can be shipped separately from the container with the intention that the instructional material and the kit be used cooperatively by the recipient.

The “stringency” with which two polynucleotides anneal means the relative likelihood that the polynucleotides will anneal in a solution as the conditions of the solution become less favorable for annealing. Examples of stringent conditions are known in the art and can be found in available references (e.g., Current Protocols in Molecular Biology, John Wiley & Sons, N.Y., 1989, 6.3.1-6.3.6). Aqueous and non-aqueous annealing methods are described in that reference and either can be used. In general, a first pair of polynucleotides anneal with higher stringency than a second pair if the first pair is more likely to anneal (or remain annealed) as one or more of the salt concentration, temperature, and detergent concentration are increased.

With respect to a disorder, a “correlation factor” for a disorder-associated polymorphism is the fractions of humans who are heterozygous or homozygous for the polymorphism who exhibit the disorder. The correlation factor can, alternatively, be based solely on those who are heterozygous, solely on those who are homozygous, or on those who are either heterozygous or homozygous.

A “non-extendable” nucleotide residue is a nucleotide residue that is capable of being added to a polynucleotide by a polymerase (i.e., by extension of the polynucleotide in association with a complement thereof, catalyzed by the polymerase) and that, upon addition to the polynucleotide, renders the polynucleotide incapable of being further extended by the polymerase.

Description

The invention relates to kits and methods for assessing the cardiovascular health of a human by assessing occurrence in the human's genome of genetic polymorphisms that are associated with disorders. Unlike other methods that predict susceptibility to cardiovascular disorders based on occurrence of a particular polymorphism in a particular gene, a panel of informative genes and polymorphisms is disclosed herein. Using two or more of the genes in this panel, one can assess the likelihood that a human is experiencing, or will likely experience, a cardiovascular disorder, regardless of whether the disorder has been specifically associated with one or more of the polymorphisms in the panel. Owing to the general predictive nature of the kits and methods disclosed herein, those kits and methods are not limited to detection of polymorphisms for which a linkage between occurrence of the polymorphism and development of a particular cardiovascular disorder has been proven.

It has been discovered a human's general state of cardiovascular health can be assessed by determining which polymorphic forms of certain genes are present in the human's genome. The genes which are assessed are disclosed herein. Assessment of disorder-associated polymorphisms in two or more of these genes (preferably four, six, eight, ten, fifteen, or twenty of these genes) in a human's genome is predictive of the human's cardiovascular health. The greater the number of these genes in which occurrence of disorder-associated polymorphisms is assessed, the greater the precision of the methods for predicting the human's cardiovascular health is likely to be.

Genes in which disorder-associated polymorphisms occur that are useful for assessing the cardiovascular health of an individual include

a) the gene which encodes apolipoprotein B;

b) the gene which encodes apolipoprotein E;

c) the gene which encodes paraoxonase 1;

d) the gene which encodes type 1 angiotensin II receptor;

e) the gene which encodes cytochrome b-245(alpha);

f) the gene which encodes prothrombin;

g) the gene which encodes coagulation factor VII;

h) the gene which encodes platelet glycoprotein 1b alpha;

i) the gene which encodes platelet glycoprotein IIIa;

j) the gene which encodes endothelial nitric oxide synthase;

k) the gene which encodes 5,10-methylene tetrahydrofolate reductase;

1) the gene which encodes angiotensinogen;

m) the gene which encodes plasminogen activator inhibitor 1;

n) the gene which encodes coagulation factor V;

o) the gene which encodes alpha adducin I;

p) the gene which encodes cytochrome P450;

q) the gene which encodes G protein beta, polypeptide 3;

r) the gene which encodes methionine synthase reductase;

s) the gene which encodes endothelial adhesion molecule 1; and

t) the gene which encodes cholesteryl ester transferase.

Occurrence in a patient's genome of substantially any (if not any) disorder-associated polymorphism that in one of genes a)-t) is an indication that the patient is at a greater risk of developing a cardiovascular disorder (if the patient is not already afflicted with the disorder) than a human whose genome does not include the disorder-associated polymorphism. Occurrence of multiple disorder-associated polymorphisms in these genes in a patient's genome is an indication that that patient is at greater risk for developing a cardiovascular disorder (i.e., has poorer cardiovascular health) than a human in whose genome fewer (or none) of the disorder-associated polymorphisms occur. Thus, there is a cumulative effect of disorder-associated polymorphisms in the genes identified herein on the cardiovascular health of the human in which they occur.

Occurrence of two copies of the same disorder-associated polymorphism in the same human (i.e., homozygosity for the polymorphism) is an indication that the human is at a greater risk for developing a cardiovascular disorder (i.e., has poorer cardiovascular health) than a human in whom only a single copy of the polymorphism occurs (i.e., an individual heterozygous for the disorder-associated polymorphism).

Although the invention is not limited to particular disorder-associated polymorphisms in the genes identified herein, it is recognized that disorder-associated polymorphisms that occur in particular portions of the genes can be more significant indicators of cardiovascular health than disorder-associated polymorphisms that occur in particular portions of the genes. Thus, disorder-associated polymorphisms that occur in the following regions of the indicated genes can be weighted more heavily than disorder-associated polymorphisms that occur in other portions of the genes. These polymorphisms include

A) disorder-associated polymorphisms that occur in the open reading frame encoding apolipoprotein B;

B) disorder-associated polymorphisms that occur in the open reading frame encoding apolipoprotein E;

C) disorder-associated polymorphisms that occur in the open reading frame encoding paraoxonase 1;

D) disorder-associated polymorphisms that occur in the 3′-untranslated region of the gene encoding type 1 angiotensin II receptor;

E) disorder-associated polymorphisms that occur in the open reading frame encoding cytochrome b-245(alpha);

F) disorder-associated polymorphisms that occur in the 3′-untranslated region of the gene encoding prothrombin;

G) disorder-associated polymorphisms that occur in the open reading frame encoding coagulation factor VII;

H) disorder-associated polymorphisms that occur in the open reading frame encoding platelet glycoprotein 1b alpha;

I) disorder-associated polymorphisms that occur in the open reading frame encoding platelet glycoprotein IIIa;

J) disorder-associated polymorphisms that occur in the open reading frame encoding endothelial nitric oxide synthase;

K) disorder-associated polymorphisms that occur in the open reading frame encoding 5, 10-methylene tetrahydrofolate reductase;

L) disorder-associated polymorphisms that occur in the open reading frame encoding angiotensinogen;

M) disorder-associated polymorphisms that occur in the promoter operably linked with the open reading frame encoding plasminogen activator inhibitor 1;

N) disorder-associated polymorphisms that occur in the open reading frame encoding coagulation factor V;

O) disorder-associated polymorphisms that occur in the open reading frame encoding alpha adducin I;

P) disorder-associated polymorphisms that occur in the promoter operably linked with the open reading frame encoding cytochrome P450;

Q) disorder-associated polymorphisms that occur in the open reading frame encoding G protein beta, polypeptide 3;

R) disorder-associated polymorphisms that occur in the open reading frame encoding methionine synthase reductase;

S) disorder-associated polymorphisms that occur in the open reading frame encoding endothelial adhesion molecule 1; and

T) disorder-associated polymorphisms that occur in the promoter operably linked with the open reading frame encoding cholesteryl ester transferase.

Occurrence of any of a number of particular polymorphisms can be assayed in order to assess an individual's cardiovascular health. A non-limiting list of such polymorphisms include the following:

I) a polymorphism manifested as occurrence of a codon encoding either glutamine or lysine at the codon including nucleotide residue 4154 of the open reading frame encoding apolipoprotein B;

II) a polymorphism manifested as occurrence of a codon encoding either arginine or glutamate at the codon including nucleotide residue 3611 of the open reading frame encoding apolipoprotein B;

III) a polymorphism manifested as occurrence of a codon encoding either arginine or cysteine at codon 112 of the open reading frame encoding apolipoprotein E;

IV) a polymorphism manifested as occurrence of a codon encoding either arginine or cysteine at codon 158 of the open reading frame encoding apolipoprotein E;

V) a polymorphism manifested as occurrence of a codon encoding either arginine or glutamine at codon 192 of the open reading frame encoding apolipoprotein E;

VI) a polymorphism manifested as occurrence of either an adenosine residue or a cytosine residue at nucleotide residue 1166 in the 3′-untranslated region of the gene encoding type 1 angiotensin II receptor;

VII) a polymorphism manifested as occurrence of a codon encoding either histidine or tyrosine at codon 72 of the open reading frame encoding cytochrome b-245(alpha);

VIII) a polymorphism manifested as occurrence of either an adenosine residue or a guanine residue at nucleotide residue 20210 in the 3′-untranslated region of the gene encoding prothrombin;

IX) a polymorphism manifested as occurrence of a codon encoding either arginine or glutamine at codon 353 of the open reading frame encoding coagulation factor VII;

X) a polymorphism manifested as occurrence of a codon encoding either threonine or methionine at codon 145 of the open reading frame encoding platelet glycoprotein 1b alpha;

XI) a polymorphism manifested as occurrence of a codon encoding either leucine or proline at codon 33 of the open reading frame encoding platelet glycoprotein IIIa;

XII) a polymorphism manifested as occurrence of a codon encoding either glutamate or aspartate at codon 298 of the open reading frame encoding endothelial nitric oxide synthase;

XIII) a polymorphism manifested as occurrence of a codon encoding either alanine or valine at codon 222 of the open reading frame encoding 5, 10-methylene tetrahydrofolate reductase;

XIV) a polymorphism manifested as occurrence of a codon encoding either methionine or threonine at codon 235 of the open reading frame encoding angiotensinogen;

XV) a polymorphism manifested as occurrence of a codon encoding either threonine or methionine at codon 174 of the open reading frame encoding angiotensinogen;

XVI) a polymorphism manifested as occurrence of either a tetra-guanine repeat or a penta-guanine repeat near nucleotide residue −675 of the promoter operably linked with the open reading frame encoding plasminogen activator inhibitor 1;

XVII) a polymorphism manifested as occurrence of a codon encoding either arginine or glutamine at codon 506 of the open reading frame encoding coagulation factor V;

XVIII) a polymorphism manifested as occurrence of a codon encoding either glycine or tryptophan at codon 460 of the open reading frame encoding alpha adducin I;

XIX) a polymorphism manifested as occurrence of either a cytosine residue or a thymine residue at nucleotide residue −344 of the promoter operably linked with the open reading frame encoding cytochrome P450;

XX) a polymorphism manifested as occurrence of either a cytosine residue or a thymine residue at nucleotide residue 825 of the open reading frame encoding G protein beta, polypeptide 3;

XXI) a polymorphism manifested as occurrence of a codon encoding either isoleucine or methionine at codon 22 of the open reading frame encoding methionine synthase reductase;

XXII) a polymorphism manifested as occurrence of a codon encoding either serine or arginine at codon 128 of the open reading frame encoding endothelial adhesion molecule 1;

XXIII) a polymorphism manifested as occurrence of either a cytosine residue or an adenosine residue at nucleotide residue −629 of the promoter operably linked with the open reading frame encoding cholesteryl ester transferase; and

XXIV) a polymorphism manifested as occurrence of either a guanine residue or an adenosine residue at nucleotide residue −971 of the promoter operably linked with the open reading frame encoding cholesteryl ester transferase.

Occurrence of a disorder-associated polymorphism in an individual's genome can be assessed in any of a variety of ways. In one embodiment, a simple test (e.g., primer extension, PCR amplification, or molecular beacon oligonucleotide-binding) is used to determine whether or not the disorder-associated polymorphism occurs in the individual's genome (i.e., without regard to copy number). In another embodiment, a test is used to determine whether the individual's genome includes a disorder-non-associated polymorphism corresponding to a known disorder-associated polymorphism in a gene disclosed herein (i.e., as an indication that the individual is at least heterozygous for the disorder-non-associated polymorphism). In yet another embodiment, a test (i.e., using multiple probes or primers) is used that is able to detect both disorder-associated polymorphisms and disorder-non-associated polymorphisms in two or more genes disclosed herein. Using such a test, one can determine both occurrence of a disorder-associated polymorphism in an individual's genome and whether the individual is homozygous or heterozygous for the disorder-associated polymorphism. This test also permits ‘checking’ of results, since it can both account for all known polymorphic forms and indicate when a previously uncharacterized polymorphism occurs at or near the site of a known polymorphism.

In a kit or method for detecting both disorder-associated polymorphisms and disorder-non-associated polymorphisms known for the genes disclosed herein, one or more (preferably at least two, four, six, eight, ten, twenty, thirty, or forty or more) of the following polymorphisms can be assessed:

i) a polymorphism manifested as occurrence of a codon encoding glutamine at the codon including nucleotide residue 4154 of the open reading frame encoding apolipoprotein B;

ii) a polymorphism manifested as occurrence of a codon encoding lysine at the codon including nucleotide residue 4154 of the open reading frame encoding apolipoprotein B;

iii) a polymorphism manifested as occurrence of a codon encoding arginine at the codon including nucleotide residue 3611 of the open reading frame encoding apolipoprotein B;

iv) a polymorphism manifested as occurrence of a codon encoding glutamate at the codon including nucleotide residue 3611 of the open reading frame encoding apolipoprotein B;

v) a polymorphism manifested as occurrence of a codon encoding arginine at codon 112 of the open reading frame encoding apolipoprotein E;

vi) a polymorphism manifested as occurrence of a codon encoding cysteine at codon 112 of the open reading frame encoding apolipoprotein E;

vii) a polymorphism manifested as occurrence of a codon encoding arginine at codon 158 of the open reading frame encoding apolipoprotein E;

viii) a polymorphism manifested as occurrence of a codon encoding cysteine at codon 158 of the open reading frame encoding apolipoprotein E;

ix) a polymorphism manifested as occurrence of a codon encoding arginine at codon 192 of the open reading frame encoding apolipoprotein E;

x) a polymorphism manifested as occurrence of a codon encoding glutamine at codon 192 of the open reading frame encoding apolipoprotein E;

xi) a polymorphism manifested as occurrence of an adenosine residue at nucleotide residue 1166 in the 3′-untranslated region of the gene encoding type 1 angiotensin II receptor;

xii) a polymorphism manifested as occurrence of a cytosine residue at nucleotide residue 1166 in the 3′-untranslated region of the gene encoding type 1 angiotensin II receptor;

xiii) a polymorphism manifested as occurrence of a codon encoding histidine at codon 72 of the open reading frame encoding cytochrome b-245(alpha);

xiv) a polymorphism manifested as occurrence of a codon encoding tyrosine at codon 72 of the open reading frame encoding cytochrome b-245(alpha);

xv) a polymorphism manifested as occurrence of an adenosine residue at nucleotide residue 20210 in the 3′-untranslated region of the gene encoding prothrombin;

xvi) a polymorphism manifested as occurrence of a guanine residue at nucleotide residue 20210 in the 3′-untranslated region of the gene encoding prothrombin;

xvii) a polymorphism manifested as occurrence of a codon encoding arginine at codon 353 of the open reading frame encoding coagulation factor VII;

xviii) a polymorphism manifested as occurrence of a codon encoding glutamine at codon 353 of the open reading frame encoding coagulation factor VII;

xix) a polymorphism manifested as occurrence of a codon encoding threonine at codon 145 of the open reading frame encoding platelet glycoprotein 1b alpha;

xx) a polymorphism manifested as occurrence of a codon encoding methionine at codon 145 of the open reading frame encoding platelet glycoprotein 1b alpha;

xxi) a polymorphism manifested as occurrence of a codon encoding leucine at codon 33 of the open reading frame encoding platelet glycoprotein IIIa;

xxii) a polymorphism manifested as occurrence of a codon encoding proline at codon 33 of the open reading frame encoding platelet glycoprotein IIIa;

xxiii) a polymorphism manifested as occurrence of a codon encoding glutamate at codon 298 of the open reading frame encoding endothelial nitric oxide synthase;

xxiv) a polymorphism manifested as occurrence of a codon encoding aspartate at codon 298 of the open reading frame encoding endothelial nitric oxide synthase;

xxv) a polymorphism manifested as occurrence of a codon encoding alanine at codon 222 of the open reading frame encoding 5, 10-methylene tetrahydrofolate reductase;

xxvi) a polymorphism manifested as occurrence of a codon encoding valine at codon 222 of the open reading frame encoding 5, 10-methylene tetrahydrofolate reductase;

xxvii) a polymorphism manifested as occurrence of a codon encoding methionine at codon 235 of the open reading frame encoding angiotensinogen;

xxviii) a polymorphism manifested as occurrence of a codon encoding threonine at codon 235 of the open reading frame encoding angiotensinogen;

xxix) a polymorphism manifested as occurrence of a codon encoding threonine at codon 174 of the open reading frame encoding angiotensinogen;

xxx) a polymorphism manifested as occurrence of a codon encoding methionine at codon 174 of the open reading frame encoding angiotensinogen;

xxxi) a polymorphism manifested as occurrence of a tetra-guanine repeat near nucleotide residue −675 of the promoter operably linked with the open reading frame encoding plasminogen activator inhibitor 1;

xxxii) a polymorphism manifested as occurrence of a penta-guanine repeat near nucleotide residue −675 of the promoter operably linked with the open reading frame encoding plasminogen activator inhibitor 1;

xxxiii) a polymorphism manifested as occurrence of a codon encoding arginine at codon 506 of the open reading frame encoding coagulation factor V;

xxxiv) a polymorphism manifested as occurrence of a codon encoding glutamine at codon 506 of the open reading frame encoding coagulation factor V;

xxxv) a polymorphism manifested as occurrence of a codon encoding glycine at codon 460 of the open reading frame encoding alpha adducin I;

xxxvi) a polymorphism manifested as occurrence of a codon encoding tryptophan at codon 460 of the open reading frame encoding alpha adducin I;

xxxvii) a polymorphism manifested as occurrence of a cytosine residue at nucleotide residue −344 of the promoter operably linked with the open reading frame encoding cytochrome P450;

xxxviii) a polymorphism manifested as occurrence of a thymine residue at nucleotide residue −344 of the promoter operably linked with the open reading frame encoding cytochrome P450;

xxxix) a polymorphism manifested as occurrence of a cytosine residue at nucleotide residue 825 of the open reading frame encoding G protein beta, polypeptide 3;

xxxx) a polymorphism manifested as occurrence of a thymine residue at nucleotide residue 825 of the open reading frame encoding G protein beta, polypeptide 3;

xxxxi) a polymorphism manifested as occurrence of a codon encoding isoleucine at codon 22 of the open reading frame encoding methionine synthase reductase;

xxxxii) a polymorphism manifested as occurrence of a codon encoding methionine at codon 22 of the open reading frame encoding methionine synthase reductase;

xxxxiii) a polymorphism manifested as occurrence of a codon encoding serine at codon 128 of the open reading frame encoding endothelial adhesion molecule 1;

xxxxiv) a polymorphism manifested as occurrence of a codon encoding arginine at codon 128 of the open reading frame encoding endothelial adhesion molecule 1;

xxxxv) a polymorphism manifested as occurrence of a cytosine residue at nucleotide residue −629 of the promoter operably linked with the open reading frame encoding cholesteryl ester transferase;

xxxxvi) a polymorphism manifested as occurrence of an adenosine residue at nucleotide residue −629 of the promoter operably linked with the open reading frame encoding cholesteryl ester transferase;

xxxxvii) a polymorphism manifested as occurrence of a guanine residue at nucleotide residue −971 of the promoter operably linked with the open reading frame encoding cholesteryl ester transferase; and

xxxxviii) a polymorphism manifested as occurrence of an adenosine residue at nucleotide residue −971 of the promoter operably linked with the open reading frame encoding cholesteryl ester transferase.

An important aspect of this invention is that human cardiovascular health (e.g., susceptibility to a CAD such as myocardial infarction, to hypertension, or to phlebothrombosis) can be associated with occurrence in the human's genome of a disorder-associated polymorphism in one of the gene described herein—even if there is no known biochemical or physiological association between occurrence of the polymorphism and incidence of the disorder. Put another way, the present inventors have discovered that genes and polymorphisms disclosed herein are predictive indicators of the general state of an individual human's cardiovascular health. By assessing whether or not disorder-associated polymorphisms occur in the genes identified herein in an individual (and how many such polymorphisms in those genes), one can predict the likelihood that the individual has, or will develop a cardiovascular disorder.

Early detection of a predisposition to develop a cardiovascular disorder can enable an individual (or the individual's physician) to take steps to delay, inhibit, alleviate (i.e., reduce the severity of), or even prevent the disorder. The appropriate steps for treating and preventing cardiovascular disorders are well known and include modifying diet, exercise, and intake of nutrients and pharmaceuticals. For particular cardiovascular disorders for which association between occurrence of the disorder and occurrence of particular polymorphisms in a patient's genome is disclosed herein, additional palliative, therapeutic, or prophylactic steps that are specific for the disorder can also be undertaken once a patient's susceptibility to the disorder is known. By way of example, association between occurrence of particular polymorphisms and phlebothrombosis is disclosed herein. Patients disposed to phlebothrombosis are often advised to avoid long distance travel by airline or train, or at least to be certain to exercise their legs during such travel. The kits and methods disclosed herein can be used to advise a potential traveler whether such exercise is likely to be beneficial during travel.

It was not previously appreciated that detection in a human's genome of two or more disorder-associated polymorphisms in the genes disclosed herein is indicative that the human globally exhibits poorer cardiovascular health, as manifested as greater susceptibility to cardiovascular disorders. Previous studies are believed to have recognized only association between a polymorphism in only individual genes identified herein and a particular disorder. The inventors believe that they are the first to describe methods and kits for assessing a human's global (i.e., not limited to a particular tissue, cell type, or organ) susceptibility to cardiovascular disorders based on occurrence in the human of certain polymorphisms that are not necessarily recognized as being associated with that disorder.

It has been discovered that disorder-associated polymorphisms in certain of the genes disclosed herein can be used to assess both an individual's overall cardiovascular health and the likelihood that the individual will develop (or is currently afflicted with) one or more particular cardiovascular disorders.

It has been discovered that disorder-associated polymorphisms that occur in the genes identified herein as a)-j), l), m), s), and t) can be used to assess both an individual's overall cardiovascular health and the likelihood that the individual will develop (or is currently afflicted with) a CAD. One such CAD with which occurrence of disorder-associated polymorphisms that occur in these genes is correlated is myocardial infarction. Thus, in one embodiment of the kits and method described herein, occurrence of disorder-associated polymorphisms (and/or disorder-non-associated polymorphisms) is assessed in two or more of the genes identified herein as a)-j), l), m), s), and t), such as occurrence of a disorder-associated polymorphisms identified herein as A)-J), L), M), S), and T). By way of example, the kit or method can involve assessing occurrence of one or more of the polymorphisms identified herein as i)-xxiv), xxvii), xxviii), xxxi), xxxii), and xxxxiii)-xxxxviii). Such kits and methods can be used to assess a patient's cardiovascular health and/or the patient's predisposition for a CAD such as myocardial infarction.

It has been discovered that disorder-associated polymorphisms that occur in the genes identified herein as d), j), l), and o)-q) can be used to assess both an individual's overall cardiovascular health and the likelihood that the individual will develop (or is currently afflicted with) hypertension. Thus, in one embodiment of the kits and method described herein, occurrence of disorder-associated polymorphisms (and/or disorder-non-associated polymorphisms) is assessed in two or more of the genes identified herein as d), j), l), and o)-q), such as occurrence of a disorder-associated polymorphisms identified herein as D), J), L), and O)-Q). By way of example, the kit or method can involve assessing occurrence of one or more of the polymorphisms identified herein as xi), xii), xxiii), xxiv), xxvii)-xxx), and xxxv)-xxxx). Such kits and methods can be used to assess a patient's cardiovascular health and/or the patient's predisposition for hypertension.

It has been discovered that disorder-associated polymorphisms that occur in the genes identified herein as e), k), n), and r) can be used to assess both an individual's overall cardiovascular health and the likelihood that the individual will develop (or is currently afflicted with) phlebothrombosis. Thus, in one embodiment of the kits and method described herein, occurrence of disorder-associated polymorphisms (and/or disorder-non-associated polymorphisms) is assessed in two or more of the genes identified herein as e), k), n), and r), such as occurrence of a disorder-associated polymorphisms identified herein as E), K), N), and R). By way of example, the kit or method can involve assessing occurrence of one or more of the polymorphisms identified herein as xiii), xiv), xxv), xxvi), xxxiii), xxxiv), xxxxi), and xxxxii). Such kits and methods can be used to assess a patient's cardiovascular health and/or the patient's predisposition for phlebothrombosis.

Methods of Assessing Cardiovascular Health

The invention includes a method of assessing the cardiovascular health (e.g., relative susceptibility to one or more cardiovascular disorders) of a human. This cardiovascular health can be calculated relative to a hypothetical human whose genome does not contain a single disorder-associated polymorphism in a gene disclosed herein. Alternatively, susceptibility can be calculated relative to another human who may have one or more different disorder-associated polymorphism than the human being assessed. In practice, the basis upon which raw susceptibility scores are calculated is immaterial, so long as the same basis is used for all humans whose scores are to be compared (i.e., so that the scores are relatable to one another).

The relative cardiovascular health of a human assessment of risks and benefits of a variety of compositions, conditions, and interventions. In one embodiment, the cardiovascular health of a human can be used to determine whether the human would benefit by supplementing nutritional intake with a composition that contains one or more cardiovascular protective agents. Numerous cardiovascular protective agents are know and additional agents are sure to be discovered in time. Examples of cardiovascular protective agents include vitamins (including anti-oxidant vitamins such as vitamins C and E), minerals (e.g., calcium), ethanol (in moderation as a dietary beverage), cholesterol- and bile acid-binding agents for lowering serum cholesterol levels, pharmaceutical agents (e.g., statins) for lowering serum cholesterol, and other cardioprotective and vascular protective pharmaceutical agents. Furthermore, the cardiovascular health of the human can indicate an appropriate dose of such an agent. In another embodiment, suitability of a condition or intervention for a human (e.g., administration to the human of dietary cholesterol-binding agents or performance of a coronary balloon angioplasty or bypass procedure) can be determined by assessing the human's cardiovascular health as described herein.

Cardiovascular health of a human is assessed by assessing occurrence in the human's genome of disorder-associated polymorphisms in a plurality of genes disclosed herein (e.g., 2, 3, 4, 6, 8, 10, 15, or 20 genes). Occurrence of a disorder-associated polymorphism in one of these genes is an indication that the human has a greater susceptibility to cardiovascular disorders (i.e., poorer cardiovascular health) than a human in whose genome the polymorphism does not occur. Of course, occurrence of two or more such polymorphisms in the human's genome indicates that the human exhibits even greater susceptibility to cardiovascular disorders (and poorer cardiovascular health).

Occurrence of every disorder-associated polymorphisms in a gene disclosed herein is not necessarily equally indicative of susceptibility to cardiovascular disorders and poorer cardiovascular health. In order to account for differences in the significance of various disorder-associated polymorphisms, a weighting factor can be assigned to each polymorphism detected in the methods and kits described herein. As indicated above, some genes have a more significant role in cardiovascular health in humans than others. All else being equal, disorder-associated polymorphisms that occur in one of these genes are more significant than polymorphisms that occur in genes having less significant roles in cardiovascular health. Thus, a greater weighting factor can be assigned to these polymorphisms than to others. By way of example, the weighting factor assigned to these polymorphisms can be 1 to 10 times greater than the weighting factor assigned to disorder-associated polymorphisms (having equal correlation with the corresponding disorder, as discussed below) in other genes.

Another factor which can influence the significance that is assigned to occurrence of a disorder-associated polymorphism in a human's genome is the degree to which the polymorphism is correlated with the corresponding disorder. Some disorders are highly correlated with occurrence of a genetic polymorphism, and other disorders exhibit lower correlation with a polymorphism. When a polymorphism is reported to be associated with a disorder (i.e., with a disease or pathological condition), a degree of correlation between the polymorphism and the disorder is often reported. One useful way of calculating a factor that describes correlation between a polymorphism and a disorder is to calculate an odds ratio that describes the likelihood that an individual in whose genome the disorder-associate polymorphism occurs will exhibit or develop the disorder. Because the kits and methods described herein can be used to detect whether the human is homozygous for the disease-associated polymorphism, odds ratios calculated for homozygous individuals can also be used, if they are available. Odds ratios can be calculated as described in the art.

For a disorder-associated polymorphism, the odds ratio can be calculated as follows. First, the odds of being afflicted with the disorder are calculated for a first population in whom the polymorphism occurs by dividing the number of afflicted individuals in the first population by the total number of individuals in the first population. Second, the odds of being afflicted with the disorder are calculated for a first population in whom the polymorphism does not occur by dividing the number of afflicted individuals in the second population by the total number of individuals in the second population. Third, the odds ratio is calculated by dividing the odds for the first population by the odds for the second population. If the odds ratio is greater than one, then this is an indication that occurrence of the polymorphism is associated with occurrence of the disorder. Furthermore, the magnitude of the odds ratio is an indication of the significance of the association.

An overall cardiovascular health score for a human can be determined as follows. A significance score can be assigned to each disorder-associated polymorphism that is detected in the human's genome using a method or kit described herein. The significance score is a constant (e.g., 1.00), and is multiplied by any significance factor (e.g., 1-10) and by any correlation factor that is available. If information is available which describes the correlation between homozygosity for the polymorphism and the corresponding disorder, then that correlation factor should be used in place of the correlation factor for mere occurrence of the polymorphism, at least if the method or kit is used to rule out occurrence in the subject's genome of corresponding disorder-non-associated polymorphisms. If significance and correlation factors are not available, then values of 1.00 should be assigned to each. An overall score is determined by summing the significance score for each disorder-associated polymorphism that is detected using the method or kit. This overall cardiovascular health score can be compared with the values obtained from other subjects, or it can be compared with the value (i.e., zero) which would be expected to occur in a human whose genome does not include any disorder-associated polymorphism in a gene disclosed herein. A high overall cardiovascular health score corresponds to poor cardiovascular health. Thus, for two individuals having different overall cardiovascular health scores, the individual having the lower score has better overall cardiovascular health than the individual having the higher score.

The method used to assess occurrence of any particular disorder-associated polymorphism (or disorder-non-associated polymorphism) is not critical. Numerous methods of detecting occurrence of a polymorphism are known in the art, and substantially any of those methods can be used in the kits and methods described herein. Naturally, the reagents included in the kit will vary depending on the method to be used to detect the polymorphisms. Examples of some suitable polymorphism detection methods are provided below.

In one embodiment, a pair of oligonucleotide primers are used to amplify a portion of the gene that includes a polymorphic region. Detection of one or more of the polymorphisms that occur at the polymorphic region can be achieved by contacting the amplified portion with an oligonucleotide having a sequence that it will anneal under stringent conditions with the amplified portion only if one polymorphism occurs at the portion, but will not anneal with the amplified portion if another polymorphism occurs at that portion. Various acceptable stringent conditions are known in the art, and can be modified by the skilled artisan as appropriate to any particular amplified portion/oligonucleotide pair. An example of stringent conditions is hybridization in 6× sodium chloride/sodium citrate (SSC) at about 45° C., followed by one or more washes in 0.2×SSC, 0.1% (w/v) SDS at 50° C.

In an alternative embodiment, one or more molecular beacon oligonucleotides are used to detect polymorphisms (disorder-associated, disorder-non-associated, or both) in a sample that contains a copy of the subject's genome, a fraction of the subject's genome, or amplification products generated from the subject's genome (e.g., amplified portions of on or more of the genes disclosed herein in which portions polymorphisms are known to occur).

Molecular beacon probes are single-stranded oligonucleotides having a fluorescent label (e.g. rhodamine, FAM, TET, VIC, JOE, or HEX) attached to the 5′-end thereof and a fluorescence quencher (e.g. TAMRA or DABCYL) attached to the 3′-end thereof (or vice versa), as described (Kostrikis et al., 1998, Science 279:1228-1229). The sequence of each molecular beacon probe is selected to include two complementary hairpin regions, whereby the probe can self-anneal to form a hairpin structure. The 5′- and 3′-ends are brought into close association when the hairpin structure forms. The probe also comprises a targeting portion which is selected to be complementary to a target sequence (e.g. a single polymorphism of a gene disclosed herein). The targeting portion and at least one of the hairpin regions are located in close proximity to one another, meaning that the targeting portion either overlaps the hairpin region or flanks it, having no more than about 5 nucleotide residues therebetween.

If the hairpin regions of the molecular beacon probe anneal with one another, then the probe does not fluoresce, because the hairpin structure forms and the fluorescence quencher attached to one end of the probe quenches fluorescence of the label attached to the other end of the probe. If the targeting portion of the probe anneals with a region of a nucleic acid having the target sequence, then formation of the hairpin structure is inhibited, the fluorescence quencher is not brought into association with the fluorescent label, and the probe fluoresces. Multiple molecular beacon probes can be used in a single reaction mixture, and fluorescence associated with the probes can be differentiated if the molecular beacon probes are spectrally distinct.

Thus, in this embodiment, one or more molecular beacon probes are used, each having targeting portion which is complementary to a target region (e.g. 20 to 40 nucleotide residues, more preferably 20 to 30 residues) of one polymorphism of a gene disclosed herein. If the polymorphism to be detected is a single nucleotide polymorphism (SNP), then the target region includes, and preferably is approximately centered around, the nucleotide residue at which the polymorphism occurs. More preferably, two such probes are used, one having a targeting region completely complementary to the target region of one polymorphism of the gene (e.g., one of two polymorphisms of an SNP), and the other having a targeting region completely complementary to the target region of a corresponding polymorphism of the gene (e.g., the other polymorphism of the SNP).

In yet another embodiment of how polymorphisms in gene disclosed herein can be assessed, oligonucleotide primers which are complementary to a region adjacent a characteristic residue of the polymorphism are extended using a polymerase enzyme, and the identity of the nucleotide residue that is added to the primer in the position complementary to the characteristic residue is determined. The primer can be extended in the presence of non-extendable nucleotide residues in order to ensure that a limited number of (or only one) nucleotide residues are incorporated into the primer. Methods of this type are known in the art (e.g., the SNP-IT® technology of Orchid Biocomputer, Inc.) and are described, for example in U.S. Pat. Nos. 6,013,431 and 6,004,744.

Methods of Assessing Susceptibility to Individual Cardiovascular Disorders

An patient's overall cardiovascular health score is predictive of the patient's susceptibility to individual cardiovascular disorders (a higher overall score indicating a greater susceptibility to such disorders). Furthermore, correlation between several particular cardiovascular disorders (e.g., hypertension, phlebothrombosis, and CADs such as myocardial infarction) and particular disorder-associated polymorphisms are disclosed herein. The rate or likelihood of development and progression of cardiovascular disorders can be estimated by assessing the overall cardiovascular health of a patient. The rate or likelihood of development and progression of the particular cardiovascular disorders disclosed herein can be estimated by assessing occurrence of the corresponding polymorphisms disclosed herein.

The individual cardiovascular disorders for which susceptibility can be assessed using these methods are not limited to those disclosed herein. The methods can be used to assess susceptibility to substantially any cardiovascular disorder. However, it is recognized that congenital cardiovascular defects which lead to development of aberrant cardiovascular structures in utero or during the first few years of life are unlikely to be associated with the disorder-associated polymorphisms described herein.

Kits for Assessing Cardiovascular Health

The invention includes a kit for assessing the cardiovascular health of a human and/or the susceptibility of the human to a particular cardiovascular disorder. The kit contains reagents for performing one or more of the methods described herein. The reagents used in certain embodiments of the methods described herein are indicated above. Reagents useful for performing those methods using a variety of alternative sample preparation and polymorphism detection methods or chemistries are apparent to the skilled artisan.

Kits for detecting polymorphisms in individual genes are known in the art, and the kit of the invention can have similar components. However, a critical feature of the kit is that it includes reagents that permit its user to detect disorder-associated polymorphisms in at least two (or at least three, four, six, eight, ten, fifteen, or twenty) genes disclosed herein.

In one embodiment, the kit includes a plurality of oligonucleotides which anneal under stringent conditions with a disorder-associated polymorphism of one of the genes, but not with a non-disorder associated-polymorphism. Each of the oligonucleotides is preferably attached to a surface in order to facilitate handling of the oligonucleotide. The oligonucleotides can be linked with a plurality of surfaces (e.g., oligonucleotides for a particular polymorphism being attached to a particle discrete from a particle to which oligonucleotides for another polymorphism are attached), or they can be attached to discrete regions of a single surface (e.g., as in the GENECHIP™ device of Affymetrix, Inc.). Annealing between individual oligonucleotides and the polymorphism corresponding thereto can be detected using standard methods. The kit can also comprise oligonucleotides that are useful as molecular beacon probes or as extendable primers.

In one embodiment, the kit further comprises a DNA collection kit or apparatus, such as that described in co-pending U.S. Pat. No. 6,291,171. Advantageously, DNA collected using the kit or apparatus can be stored or archived, and subjected to additional testing as previously unknown disorder-associated polymorphisms are discovered in the genes disclosed herein, or as the significance of previously unappreciated polymorphisms is realized.

The invention also relates to a method of assessing the advisability that a human should employ a nutritional product comprising a cardiovascular protective agent such as those described above. The method is performed as described herein for assessing the cardiovascular health of a human. If poorer cardiovascular health is detected in the human (i.e., relative to a human not having a disorder-associated polymorphism in a gene identified herein), then it is more advisable the human should employ a nutritional product comprising the cardiovascular protective agent. A greater overall cardiovascular health score (i.e., corresponding to poorer cardiovascular health) in a human correlates with an increased advisability that the human should use, such a nutritional product, and also indicates that a greater dose of the cardiovascular agent(s) should be included in the nutritional product.

It will be appreciated by those skilled in the art that changes can made to the embodiments described above without departing from the broad inventive concept thereof.

This invention is not limited to the particular embodiments disclosed, and includes modifications within the spirit and scope of the present invention as defined by the appended claims.

What is claimed is:

1. A method of assessing cardiovascular health in a human, the method comprising assessing occurrence in the human's genome of disorder-associated polymorphisms in at least two genes selected from the group consisting of
a) the gene which encodes apolipoprotein B;
b) the gene which encodes apolipoprotein E;
c) the gene which encodes paraoxonase 1;
d) the gene which encodes type 1 angiotensin II receptor;
e) the gene which encodes cytochrome b-245(alpha);
f) the gene which encodes prothrombin;
g) the gene which encodes coagulation factor VII;
h) the gene which encodes platelet glycoprotein 1b alpha;
i) the gene which encodes platelet glycoprotein IIIa;
j) the gene which encodes endothelial nitric oxide synthase;
k) the gene which encodes 5,10-methylene tetrahydrofolate reductase;
l) the gene which encodes angiotensinogen;
m) the gene which encodes plasminogen activator inhibitor 1;
n) the gene which encodes coagulation factor V;
o) the gene which encodes alpha adducin I;
p) the gene which encodes cytochrome P450;
q) the gene which encodes G protein beta, polypeptide 3;
r) the gene which encodes methionine synthase reductase;
s) the gene which encodes endothelial adhesion molecule 1; and
t) the gene which encodes cholesteryl ester transferase,
whereby occurrence of any of the polymorphisms is an indication that the human has poorer cardiovascular health than a human whose genome does not comprise the polymorphism, and whereby occurrence of a plurality of the polymorphisms is an indication that the human has even poorer cardiovascular health than a human whose genome does not comprise the polymorphisms.

2. The method of claim 1, wherein the genes are selected from the group consisting of a)-j), l), m), s), and t).

3. The method of claim 1, wherein the genes are selected from the group consisting of d), j), l), and o)-q).

4. The method of claim 1, wherein the genes are selected from the group consisting of e), k), n), and r).

5. The method of claim 1, comprising assessing occurrence of at least two disorder-associated polymorphisms selected from the group consisting of
A) a polymorphism in the open reading frame encoding apolipoprotein B;
B) a polymorphism in the open reading frame encoding apolipoprotein E;
C) a polymorphism in the open reading frame encoding paraoxonase 1;
D) a polymorphism in the 3′-untranslated region of the gene encoding type 1 angiotensin II receptor;
E) a polymorphism in the open reading frame encoding cytochrome b-245(alpha);
F) a polymorphism in the 3′-untranslated region of the gene encoding prothrombin;
G) a polymorphism in the open reading frame encoding coagulation factor VII;
H) a polymorphism in the open reading frame encoding platelet glycoprotein 1b alpha;
I) a polymorphism in the open reading frame encoding platelet glycoprotein IIIa;
J) a polymorphism in the open reading frame encoding endothelial nitric oxide synthase;
K) a polymorphism in the open reading frame encoding 5, 10-methylene tetrahydrofolate reductase;
L) a polymorphism in the open reading frame encoding angiotensinogen;
M) a polymorphism in the promoter operably linked with the open reading frame encoding plasminogen activator inhibitor 1;
N) a polymorphism in the open reading frame encoding coagulation factor V;
O) a polymorphism in the open reading frame encoding alpha adducin I;
P) a polymorphism in the promoter operably linked with the open reading frame encoding cytochrome P450;
Q) a polymorphism in the open reading frame encoding G protein beta, polypeptide 3;
R) a polymorphism in the open reading frame encoding methionine synthase reductase;
S) a polymorphism in the open reading frame encoding endothelial adhesion molecule 1; and
T) a polymorphism in the promoter operably linked with the open reading frame encoding cholesteryl ester transferase.

6. The method of claim 5, wherein the genes are selected from the group consisting of A)-J), L), M), S), and T).

7. The method of claim 5, wherein the genes are selected from the group consisting of D), J), L), and O)-Q).

8. The method of claim 5, wherein the genes are selected from the group consisting of E), K), N), and R).

9. The method of claim 1, comprising assessing occurrence of a first polymorphism selected from the group consisting of
i) a polymorphism manifested as occurrence of a codon encoding glutamine at the codon including nucleotide residue 4154 of the open reading frame encoding apolipoprotein B;
ii) a polymorphism manifested as occurrence of a codon encoding lysine at the codon including nucleotide residue 4154 of the open reading frame encoding apolipoprotein B;
iii) a polymorphism manifested as occurrence of a codon encoding arginine at the codon including nucleotide residue 3611 of the open reading frame encoding apolipoprotein B;
iv) a polymorphism manifested as occurrence of a codon encoding glutamate at the codon including nucleotide residue 3611 of the open reading frame encoding apolipoprotein B;
v) a polymorphism manifested as occurrence of a codon encoding arginine at codon 112 of the open reading frame encoding apolipoprotein E;
vi) a polymorphism manifested as occurrence of a codon encoding cysteine at codon 112 of the open reading frame encoding apolipoprotein E;
vii) a polymorphism manifested as occurrence of a codon encoding arginine at codon 158 of the open reading frame encoding apolipoprotein E;
viii) a polymorphism manifested as occurrence of a codon encoding cysteine at codon 158 of the open reading frame encoding apolipoprotein E;
ix) a polymorphism manifested as occurrence of a codon encoding arginine at codon 192 of the open reading frame encoding apolipoprotein E;
x) a polymorphism manifested as occurrence of a codon encoding glutamine at codon 192 of the open reading frame encoding apolipoprotein E;
xi) a polymorphism manifested as occurrence of an adenosine residue at nucleotide residue 1166 in the 3′-untranslated region of the gene encoding type 1 angiotensin II receptor;
xii) a polymorphism manifested as occurrence of a cytosine residue at nucleotide residue 1166 in the 3′-untranslated region of the gene encoding type 1 angiotensin II receptor;
xiii) a polymorphism manifested as occurrence of a codon encoding histidine at codon 72 of the open reading frame encoding cytochrome b-245(alpha);
xiv) a polymorphism manifested as occurrence of a codon encoding tyrosine at codon 72 of the open reading frame encoding cytochrome b-245(alpha);
xv) a polymorphism manifested as occurrence of an adenosine residue at nucleotide residue 20210 in the 3′-untranslated region of the gene encoding prothrombin;
xvi) a polymorphism manifested as occurrence of a guanine residue at nucleotide residue 20210 in the 3′-untranslated region of the gene encoding prothrombin;
xvii) a polymorphism manifested as occurrence of a codon encoding arginine at codon 353 of the open reading frame encoding coagulation factor VII;
xviii) a polymorphism manifested as occurrence of a codon encoding glutamine at codon 353 of the open reading frame encoding coagulation factor VII;
xix) a polymorphism manifested as occurrence of a codon encoding threonine at codon 145 of the open reading frame encoding platelet glycoprotein 1b alpha;
xx) a polymorphism manifested as occurrence of a codon encoding methionine at codon 145 of the open reading frame encoding platelet glycoprotein 1b alpha;
xxi) a polymorphism manifested as occurrence of a codon encoding leucine at codon 33 of the open reading frame encoding platelet glycoprotein IIIa;
xxii) a polymorphism manifested as occurrence of a codon encoding proline at codon 33 of the open reading frame encoding platelet glycoprotein IIIa;
xxiii) a polymorphism manifested as occurrence of a codon encoding glutamate at codon 298 of the open reading frame encoding endothelial nitric oxide synthase;
xxiv) a polymorphism manifested as occurrence of a codon encoding aspartate at codon 298 of the open reading frame encoding endothelial nitric oxide synthase;
xxv) a polymorphism manifested as occurrence of a codon encoding alanine at codon 222 of the open reading frame encoding 5, 10-methylene tetrahydrofolate reductase;
xxvi) a polymorphism manifested as occurrence of a codon encoding valine at codon 222 of the open reading frame encoding 5, 10-methylene tetrahydrofolate reductase;
xxvii) a polymorphism manifested as occurrence of a codon encoding methionine at codon 235 of the open reading frame encoding angiotensinogen;
xxviii) a polymorphism manifested as occurrence of a codon encoding threonine at codon 235 of the open reading frame encoding angiotensinogen;
xxix) a polymorphism manifested as occurrence of a codon encoding threonine at codon 174 of the open reading frame encoding angiotensinogen;
xxx) a polymorphism manifested as occurrence of a codon encoding methionine at codon 174 of the open reading frame encoding angiotensinogen;
xxxi) a polymorphism manifested as occurrence of a tetra-guanine repeat near nucleotide residue −675 of the promoter operably linked with the open reading frame encoding plasminogen activator inhibitor 1;
xxxii) a polymorphism manifested as occurrence of a penta-guanine repeat near nucleotide residue −675 of the promoter operably linked with the open reading frame encoding plasminogen activator inhibitor 1;
xxxiii) a polymorphism manifested as occurrence of a codon encoding arginine at codon 506 of the open reading frame encoding coagulation factor V;
xxxiv) a polymorphism manifested as occurrence of a codon encoding glutamine at codon 506 of the open reading frame encoding coagulation factor V;
xxxv) a polymorphism manifested as occurrence of a codon encoding glycine at codon 460 of the open reading frame encoding alpha adducin I;
xxxvi) a polymorphism manifested as occurrence of a codon encoding tryptophan at codon 460 of the open reading frame encoding alpha adducin I;
xxxvii) a polymorphism manifested as occurrence of a cytosine residue at nucleotide residue −344 of the promoter operably linked with the open reading frame encoding cytochrome P450;
xxxviii) a polymorphism manifested as occurrence of a thymine residue at nucleotide residue −344 of the promoter operably linked with the open reading frame encoding cytochrome P450;
xxxix) a polymorphism manifested as occurrence of a cytosine residue at nucleotide residue 825 of the open reading frame encoding G protein beta, polypeptide 3;
xxxx) a polymorphism manifested as occurrence of a thymine residue at nucleotide residue 825 of the open reading frame encoding G protein beta, polypeptide 3;
xxxxi) a polymorphism manifested as occurrence of a codon encoding isoleucine at codon 22 of the open reading frame encoding methionine synthase reductase;
xxxxii) a polymorphism manifested as occurrence of a codon encoding methionine at codon 22 of the open reading frame encoding methionine synthase reductase;
xxxxiii) a polymorphism manifested as occurrence of a codon encoding serine at codon 128 of the open reading frame encoding endothelial adhesion molecule 1;
xxxxiv) a polymorphism manifested as occurrence of a codon encoding arginine at codon 128 of the open reading frame encoding endothelial adhesion molecule 1;
xxxxv) a polymorphism manifested as occurrence of a cytosine residue at nucleotide residue −629 of the promoter operably linked with the open reading frame encoding cholesteryl ester transferase;
xxxxvi) a polymorphism manifested as occurrence of an adenosine residue at nucleotide residue −629 of the promoter operably linked with the open reading frame encoding cholesteryl ester transferase;
xxxxvii) a polymorphism manifested as occurrence of a guanine residue at nucleotide residue −971 of the promoter operably linked with the open reading frame encoding cholesteryl ester transferase; and
xxxxviii) a polymorphism manifested as occurrence of an adenosine residue at nucleotide residue −971 of the promoter operably linked with the open reading frame encoding cholesteryl ester transferase.

10. The method of claim 9, wherein the first polymorphism is selected from the group consisting of i)-xxiv), xxvii), xxviii), xxxi), xxxii), and xxxxiii)-xxxxviii).

11. The method of claim 9, wherein the genes are selected from the group consisting of xi), xii), xxiii), xxiv), xxvii)-xxx), and xxxv)-xxxx).

12. The method of claim 9, wherein the genes are selected from the group consisting of xiii), xiv), xxv), xxvi), xxxiii), xxxiv), xxxxi), and xxxxii).

13. The method of claim 9, comprising assessing occurrence in the human's genome of disorder-associated polymorphisms in at least four of i) through xxxxviii).

14. The method of claim 9, comprising assessing occurrence in the human's genome of at least six of i) through xxxxviii).

15. The method of claim 9, comprising assessing occurrence in the human's genome of at least ten of i) through xxxxviii).

16. The method of claim 9, comprising assessing occurrence in the human's genome of at least fifteen of i) through xxxxviii).

17. The method of claim 9, comprising assessing occurrence in the human's genome of each of i) through xxxxviii).

18. The method of claim 1, wherein occurrence of an individual disorder-associated polymorphism is assessed by
contacting a nucleic acid derived from the human's genome with a first oligonucleotide that anneals with higher stringency with the disorder-associated polymorphism than with a corresponding non-disorder-associated polymorphism and
assessing annealing of the first oligonucleotide and the nucleic acid, whereby annealing of the first oligonucleotide and the nucleic acid is an indication that the human's genome comprises the disorder-associated polymorphism.

19. The method of claim 18, wherein the first oligonucleotide is attached to a support.

20. The method of claim 19, wherein the support has a plurality of different first oligonucleotides attached thereto.

21. The method of claim 19, wherein the support has attached thereto at least five first oligonucleotides that anneal with higher stringency with the disorder-associated polymorphisms than with the corresponding non-disorder-associated polymorphisms.

22. The method of claim 19, wherein the support has attached thereto at least ten first oligonucleotides that anneal with higher stringency with the disorder-associated polymorphisms than with the corresponding non-disorder-associated polymorphisms.

23. The method of claim 19, wherein the support has attached thereto at least fifteen first oligonucleotides that anneal with higher stringency with the disorder-associated polymorphisms than with the corresponding non-disorder-associated polymorphisms.

24. The method of claim 18, wherein the first oligonucleotide is a molecular beacon oligonucleotide.

25. The method of claim 18, wherein occurrence of an individual disorder-associated polymorphism is further assessed by
contacting the nucleic acid with a second oligonucleotide that anneals with higher stringency with a non-disorder-associated polymorphism than with the corresponding non-disorder-associated polymorphism and
assessing annealing of the second oligonucleotide and the nucleic acid, whereby annealing of the second oligonucleotide and the nucleic acid is an indication that the human's genome does not comprise the disorder-associated polymorphism.

26. The method of claim 25, wherein the second oligonucleotide is attached to a support.

27. The method of claim 26, wherein the first and second oligonucleotides are attached to the same support.

28. The method of claim 25, wherein the second oligonucleotide is a molecular beacon oligonucleotide.

29. The method of claim 28, wherein the first and second oligonucleotides are spectrally distinct molecular beacon oligonucleotides.

30. The method of claim 1, further comprising calculating a cardiovascular health score by summing, for each of the selected genes in which a disorder-associated polymorphism occurs in the human's genome, the product of a constant and a correlation factor, wherein the correlation factor represents the fraction of humans heterozygous or homozygous for the disorder-associated polymorphism who exhibit the corresponding disorder, whereby the cardiovascular health score represents the relative susceptibility of the human to a cardiovascular disorder.

31. The method of claim 30, wherein the same constant is used for each selected gene.

32. The method of claim 1, wherein each of the disorder-associated polymorphisms for which occurrence is assessed is a single nucleotide polymorphism (SNP).

33. The method of claim 1, wherein occurrence of a SNP is assessed by annealing a nucleic acid derived from the human's genome with a primer that is complementary to the region adjacent the SNP on its 3′ side, extending the primer using a polymerase in order to add a nucleotide residue complementary to the SNP to the primer, and detecting the identity of the nucleotide residue complementary to the SNP.

34. The method of claim 33, wherein the nucleotide residue is a non-extendable residue.

35. A method of assessing the likelihood that a human will develop a cardiovascular disorder, the method comprising assessing occurrence in the human's genome of disorder-associated polymorphisms in at least two genes selected from the group consisting of
a) the gene which encodes apolipoprotein B;
b) the gene which encodes apolipoprotein E;
c) the gene which encodes paraoxonase 1;
d) the gene which encodes type 1 angiotensin II receptor;
e) the gene which encodes cytochrome b-245(alpha);
f) the gene which encodes prothrombin;
g) the gene which encodes coagulation factor VII;
h) the gene which encodes platelet glycoprotein 1b alpha;
i) the gene which encodes platelet glycoprotein IIIa;
j) the gene which encodes endothelial nitric oxide synthase;
k) the gene which encodes 5,10-methylene tetrahydrofolate reductase;
l) the gene which encodes angiotensinogen;
m) the gene which encodes plasminogen activator inhibitor 1;
n) the gene which encodes coagulation factor V;
o) the gene which encodes alpha adducin I;
p) the gene which encodes cytochrome P450;
q) the gene which encodes G protein beta, polypeptide 3;
r) the gene which encodes methionine synthase reductase;
s) the gene which encodes endothelial adhesion molecule 1; and
t) the gene which encodes cholesteryl ester transferase,
whereby occurrence of any of the polymorphisms is an indication that the human is more susceptible to the cardiovascular disorder than a human whose genome does not comprise the polymorphism, and whereby occurrence of a plurality of the polymorphisms is an indication that the human is even more susceptible to the cardiovascular disorder than a human whose genome does not comprise the polymorphisms.

36. The method of claim 35, comprising assessing occurrence of at least two disorder-associated polymorphisms selected from the group consisting of
A) a polymorphism in the open reading frame encoding apolipoprotein B;
B) a polymorphism in the open reading frame encoding apolipoprotein E;
C) a polymorphism in the open reading frame encoding paraoxonase 1;
D) a polymorphism in the 3′-untranslated region of the gene encoding type 1 angiotensin II receptor;
E) a polymorphism in the open reading frame encoding cytochrome b-245(alpha);
F) a polymorphism in the 3′-untranslated region of the gene encoding prothrombin;
G) a polymorphism in the open reading frame encoding coagulation factor VII;
H) a polymorphism in the open reading frame encoding platelet glycoprotein 1b alpha;
I) a polymorphism in the open reading frame encoding platelet glycoprotein IIIa;
J) a polymorphism in the open reading frame encoding endothelial nitric oxide synthase;
K) a polymorphism in the open reading frame encoding 5, 10-methylene tetrahydrofolate reductase;
L) a polymorphism in the open reading frame encoding angiotensinogen;
M) a polymorphism in the promoter operably linked with the open reading frame encoding plasminogen activator inhibitor 1;
N) a polymorphism in the open reading frame encoding coagulation factor V;
O) a polymorphism in the open reading frame encoding alpha adducin I;
P) a polymorphism in the promoter operably linked with the open reading frame encoding cytochrome P450;
Q) a polymorphism in the open reading frame encoding G protein beta, polypeptide 3;
R) a polymorphism in the open reading frame encoding methionine synthase reductase;
S) a polymorphism in the open reading frame encoding endothelial adhesion molecule 1; and
T) a polymorphism in the promoter operably linked with the open reading frame encoding cholesteryl ester transferase.

37. The method of claim 35, comprising assessing occurrence of a first polymorphism selected from the group consisting of
i) a polymorphism manifested as occurrence of a codon encoding glutamine at the codon including nucleotide residue 4154 of the open reading frame encoding apolipoprotein B;
ii) a polymorphism manifested as occurrence of a codon encoding lysine at the codon including nucleotide residue 4154 of the open reading frame encoding apolipoprotein B;
iii) a polymorphism manifested as occurrence of a codon encoding arginine at the codon including nucleotide residue 3611 of the open reading frame encoding apolipoprotein B;
iv) a polymorphism manifested as occurrence of a codon encoding glutamate at the codon including nucleotide residue 3611 of the open reading frame encoding apolipoprotein B;
v) a polymorphism manifested as occurrence of a codon encoding arginine at codon 112 of the open reading frame encoding apolipoprotein E;
vi) a polymorphism manifested as occurrence of a codon encoding cysteine at codon 112 of the open reading frame encoding apolipoprotein E;
vii) a polymorphism manifested as occurrence of a codon encoding arginine at codon 158 of the open reading frame encoding apolipoprotein E;
viii) a polymorphism manifested as occurrence of a codon encoding cysteine at codon 158 of the open reading frame encoding apolipoprotein E;
ix) a polymorphism manifested as occurrence of a codon encoding arginine at codon 192 of the open reading frame encoding apolipoprotein E;
x) a polymorphism manifested as occurrence of a codon encoding glutamine at codon 192 of the open reading frame encoding apolipoprotein E;
xi) a polymorphism manifested as occurrence of an adenosine residue at nucleotide residue 1166 in the 3′-untranslated region of the gene encoding type 1 angiotensin II receptor;
xii) a polymorphism manifested as occurrence of a cytosine residue at nucleotide residue 1166 in the 3′-untranslated region of the gene encoding type I angiotensin II receptor;
xiii) a polymorphism manifested as occurrence of a codon encoding histidine at codon 72 of the open reading frame encoding cytochrome b-245(alpha);
xiv) a polymorphism manifested as occurrence of a codon encoding tyrosine at codon 72 of the open reading frame encoding cytochrome b-245(alpha);
xv) a polymorphism manifested as occurrence of an adenosine residue at nucleotide residue 20210 in the 3′-untranslated region of the gene encoding prothrombin;
xvi) a polymorphism manifested as occurrence of a guanine residue at nucleotide residue 20210 in the 3′-untranslated region of the gene encoding prothrombin;
xvii) a polymorphism manifested as occurrence of a codon encoding arginine at codon 353 of the open reading frame encoding coagulation factor VII;
xviii) a polymorphism manifested as occurrence of a codon encoding glutamine at codon 353 of the open reading frame encoding coagulation factor VII;
xix) a polymorphism manifested as occurrence of a codon encoding threonine at codon 145 of the open reading frame encoding platelet glycoprotein 1b alpha;
xx) a polymorphism manifested as occurrence of a codon encoding methionine at codon 145 of the open reading frame encoding platelet glycoprotein 1b alpha;
xxi) a polymorphism manifested as occurrence of a codon encoding leucine at codon 33 of the open reading frame encoding platelet glycoprotein IIIa;
xxii) a polymorphism manifested as occurrence of a codon encoding proline at codon 33 of the open reading frame encoding platelet glycoprotein IIIa;
xxiii) a polymorphism manifested as occurrence of a codon encoding glutamate at codon 298 of the open reading frame encoding endothelial nitric oxide synthase;
xxiv) a polymorphism manifested as occurrence of a codon encoding aspartate at codon 298 of the open reading frame encoding endothelial nitric oxide synthase;
xxv) a polymorphism manifested as occurrence of a codon encoding alanine at codon 222 of the open reading frame encoding 5, 10-methylene tetrahydrofolate reductase;
xxvi) a polymorphism manifested as occurrence of a codon encoding valine at codon 222 of the open reading frame encoding 5, 10-methylene tetrahydrofolate reductase;
xxvii) a polymorphism manifested as occurrence of a codon encoding methionine at codon 235 of the open reading frame encoding angiotensinogen;
xxviii) a polymorphism manifested as occurrence of a codon encoding threonine at codon 235 of the open reading frame encoding angiotensinogen;
xxix) a polymorphism manifested as occurrence of a codon encoding threonine at codon 174 of the open reading frame encoding angiotensinogen;
xxx) a polymorphism manifested as occurrence of a codon encoding methionine at codon 174 of the open reading frame encoding angiotensinogen;
xxxi) a polymorphism manifested as occurrence of a tetra-guanine repeat near nucleotide residue −675 of the promoter operably linked with the open reading frame encoding plasminogen activator inhibitor 1;
xxxii) a polymorphism manifested as occurrence of a penta-guanine repeat near nucleotide residue −675 of the promoter operably linked with the open reading frame encoding plasminogen activator inhibitor 1;
xxxiii) a polymorphism manifested as occurrence of a codon encoding arginine at codon 506 of the open reading frame encoding coagulation factor V;
xxxiv) a polymorphism manifested as occurrence of a codon encoding glutamine at codon 506 of the open reading frame encoding coagulation factor V;
xxxv) a polymorphism manifested as occurrence of a codon encoding glycine at codon 460 of the open reading frame encoding alpha adducin I;
xxxvi) a polymorphism manifested as occurrence of a codon encoding tryptophan at codon 460 of the open reading frame encoding alpha adducin I;
xxxvii) a polymorphism manifested as occurrence of a cytosine residue at nucleotide residue −344 of the promoter operably linked with the open reading frame encoding cytochrome P450;
xxxviii) a polymorphism manifested as occurrence of a thymine residue at nucleotide residue −344 of the promoter operably linked with the open reading frame encoding cytochrome P450;
xxxix) a polymorphism manifested as occurrence of a cytosine residue at nucleotide residue 825 of the open reading frame encoding G protein beta, polypeptide 3;
xxxx) a polymorphism manifested as occurrence of a thymine residue at nucleotide residue 825 of the open reading frame encoding G protein beta, polypeptide 3;
xxxxi) a polymorphism manifested as occurrence of a codon encoding isoleucine at codon 22 of the open reading frame encoding methionine synthase reductase;
xxxxii) a polymorphism manifested as occurrence of a codon encoding methionine at codon 22 of the open reading frame encoding methionine synthase reductase;
xxxxiii) a polymorphism manifested as occurrence of a codon encoding serine at codon 128 of the open reading frame encoding endothelial adhesion molecule 1;
xxxxiv) a polymorphism manifested as occurrence of a codon encoding arginine at codon 128 of the open reading frame encoding endothelial adhesion molecule 1;
xxxxv) a polymorphism manifested as occurrence of a cytosine residue at nucleotide residue −629 of the promoter operably linked with the open reading frame encoding cholesteryl ester transferase;
xxxxvi) a polymorphism manifested as occurrence of an adenosine residue at nucleotide residue −629 of the promoter operably linked with the open reading frame encoding cholesteryl ester transferase;
xxxxvii) a polymorphism manifested as occurrence of a guanine residue at nucleotide residue −971 of the promoter operably linked with the open reading frame encoding cholesteryl ester transferase; and
xxxxviii) a polymorphism manifested as occurrence of an adenosine residue at nucleotide residue −971 of the promoter operably linked with the open reading frame encoding cholesteryl ester transferase.

38. The method of claim 35, wherein the cardiovascular disorder is a coronary artery disease and wherein the genes are selected from the group consisting of a)-j), l), m), s), and t).

39. The method of claim 36, wherein the cardiovascular disorder is a coronary artery disease and wherein the genes are selected from the group consisting of A)-J), L), M), S), and T).

40. The method of claim 37, wherein the cardiovascular disorder is a coronary artery disease and wherein the first polymorphism is selected from the group consisting of i)-xxiv), xxvii), xxviii), xxxi), xxxii), and xxxxiii)-xxxxviii).

41. The method of claim 35, wherein the cardiovascular disorder is myocardial infarction and wherein the genes are selected from the group consisting of a)-j), l), m), s), and t).

42. The method of claim 36, wherein the cardiovascular disorder is myocardial infarction and wherein the genes are selected from the group consisting of A)-J), L), M), S), and T).

43. The method of claim 37, wherein the cardiovascular disorder is myocardial infarction and wherein the first polymorphism is selected from the group consisting of i)-xxiv), xxvii), xxviii), xxxi), xxxii), and xxxxiii)-xxxxviii).

44. The method of claim 35, wherein the cardiovascular disorder is hypertension and wherein the genes are selected from the group consisting of d), j),1), and o)-q).

45. The method of claim 36, wherein the cardiovascular disorder is hypertension and wherein the genes are selected from the group consisting of D), J), L), and O)-Q)

46. The method of claim 37, wherein the cardiovascular disorder is hypertension and wherein the genes are selected from the group consisting of xi), xii), xxiii), xxiv), xxvii)-xxx), and xxxv)-xxxx).

47. The method of claim 35, wherein the cardiovascular disorder is phlebothrombosis and wherein the genes are selected from the group consisting of e), k), n), and r).

48. The method of claim 36, wherein the cardiovascular disorder is phlebothrombosis and wherein the genes are selected from the group consisting of E), K), N), and R).

49. The method of claim 37, wherein the genes are selected from the group consisting of xiii), xiv), xxv), xxvi), xxxiii), xxxiv), xxxxi), and xxxxii).

50. A method of selecting a dose of a cardiovascular protective composition for administration to a human, the method comprising assessing occurrence in the human's genome of disorder-associated polymorphisms in at least two genes selected from the group consisting of
a) the gene which encodes apolipoprotein B;
b) the gene which encodes apolipoprotein E;
c) the gene which encodes paraoxonase 1;
d) the gene which encodes type 1 angiotensin II receptor;
e) the gene which encodes cytochrome b-245(alpha);
f) the gene which encodes prothrombin;
g) the gene which encodes coagulation factor VII;
h) the gene which encodes platelet glycoprotein 1b alpha;
i) the gene which encodes platelet glycoprotein IIIa;
j) the gene which encodes endothelial nitric oxide synthase;
k) the gene which encodes 5,10-methylene tetrahydrofolate reductase;
l) the gene which encodes angiotensinogen;
m) the gene which encodes plasminogen activator inhibitor 1;
n) the gene which encodes coagulation factor V;
o) the gene which encodes alpha adducin I;
p) the gene which encodes cytochrome P450;
q) the gene which encodes G protein beta, polypeptide 3;
r) the gene which encodes methionine synthase reductase;
s) the gene which encodes endothelial adhesion molecule 1; and
t) the gene which encodes cholesteryl ester transferase,
whereby occurrence of any of the polymorphisms is an indication that a greater dose of the composition should be administered to the human; and
selecting a dose of the composition based on occurrence of the polymorphisms.

51. A kit for assessing relative susceptibility of a human to a cardiovascular disorder, the kit comprising reagents for assessing occurrence in the human's genome of disorder-associated polymorphisms in at least two genes selected from the group consisting of
a) the gene which encodes apolipoprotein B;
b) the gene which encodes apolipoprotein E;
c) the gene which encodes paraoxonase 1;
d) the gene which encodes type 1 angiotensin II receptor;
e) the gene which encodes cytochrome b-245(alpha);
f) the gene which encodes prothrombin;
g) the gene which encodes coagulation factor VII;
h) the gene which encodes platelet glycoprotein 1b alpha;
i) the gene which encodes platelet glycoprotein IIIa;
j) the gene which encodes endothelial nitric oxide synthase;
k) the gene which encodes 5,10-methylene tetrahydrofolate reductase;
l) the gene which encodes angiotensinogen;
m) the gene which encodes plasminogen activator inhibitor 1;
n) the gene which encodes coagulation factor V;
o) the gene which encodes alpha adducin I;
p) the gene which encodes cytochrome P450;
q) the gene which encodes G protein beta, polypeptide 3;
r) the gene which encodes methionine synthase reductase;
s) the gene which encodes endothelial adhesion molecule 1; and
t) the gene which encodes cholesteryl ester transferase.

52. The kit of claim 51, wherein the reagents comprise first oligonucleotides that anneal with higher stringency with the disorder-associated polymorphisms than with corresponding non-disorder-associated polymorphisms.

53. The kit of claim 52, wherein each of the first oligonucleotides is attached to a support.

54. The kit of claim 53, wherein each of the first oligonucleotides is attached to the same support.

55. The kit of claim 53, wherein each of the first oligonucleotides is attached to a different support.

56. The kit of claim 52, wherein the first oligonucleotides are molecular beacon oligonucleotides.

57. The kit of claim 52, wherein the kit further comprises second oligonucleotides that anneal with higher stringency with the non-disorder-associated polymorphisms than with corresponding disorder-associated polymorphisms.

58. The kit of claim 57, wherein the first and second oligonucleotides are spectrally distinct molecular beacon oligonucleotide pairs.

59. The kit of claim 51, wherein the reagents comprise primers that are complementary to the region adjacent a characteristic residue of the disorder-associated polymorphism for amplifying at least the characteristic residue.

60. The kit of claim 59, further comprising a polymerase capable of extending the primers by adding a nucleotide residue complementary to the characteristic residue.

61. The kit of claim 60, further comprising a non-extendable nucleotide residue.

62. The kit of claim 51, further comprising an instructional material which includes a numerical value representing the product of a constant and a correlation factor, wherein the correlation factor represents the fraction of humans heterozygous or homozygous for the disorder-associated polymorphism who exhibit the corresponding disorder.

63. The kit of claim 62, wherein the same constant is used for each selected gene.

64. The kit of claim 51, wherein the kit comprises reagents for assessing occurrence in the human's genome of at least two polymorphisms selected from the group consisting of
I) a polymorphism manifested as occurrence of a codon encoding either glutamine or lysine at the codon including nucleotide residue 4154 of the open reading frame encoding apolipoprotein B;
II) a polymorphism manifested as occurrence of a codon encoding either arginine or glutamate at the codon including nucleotide residue 3611 of the open reading frame encoding apolipoprotein B;
III) a polymorphism manifested as occurrence of a codon encoding either arginine or cysteine at codon 112 of the open reading frame encoding apolipoprotein E;
IV) a polymorphism manifested as occurrence of a codon encoding either arginine or cysteine at codon 158 of the open reading frame encoding apolipoprotein E;
V) a polymorphism manifested as occurrence of a codon encoding either arginine or glutamine at codon 192 of the open reading frame encoding apolipoprotein E;
VI) a polymorphism manifested as occurrence of either an adenosine residue or a cytosine residue at nucleotide residue 1166 in the 3′-untranslated region of the gene encoding type 1 angiotensin II receptor;
VII) a polymorphism manifested as occurrence of a codon encoding either histidine or tyrosine at codon 72 of the open reading frame encoding cytochrome b-245(alpha);
VIII) a polymorphism manifested as occurrence of either an adenosine residue or a guanine residue at nucleotide residue 20210 in the 3′-untranslated region of the gene encoding prothrombin;
IX) a polymorphism manifested as occurrence of a codon encoding either arginine or glutamine at codon 353 of the open reading frame encoding coagulation factor VII;
X) a polymorphism manifested as occurrence of a codon encoding either threonine or methionine at codon 145 of the open reading frame encoding platelet glycoprotein 1b alpha;
XI) a polymorphism manifested as occurrence of a codon encoding either leucine or proline at codon 33 of the open reading frame encoding platelet glycoprotein IIIa;
XII) a polymorphism manifested as occurrence of a codon encoding either glutamate or aspartate at codon 298 of the open reading frame encoding endothelial nitric oxide synthase;
XIII) a polymorphism manifested as occurrence of a codon encoding either alanine or valine at codon 222 of the open reading frame encoding 5, 10-methylene tetrahydrofolate reductase;
XIV) a polymorphism manifested as occurrence of a codon encoding either methionine or threonine at codon 235 of the open reading frame encoding angiotensinogen;
XV) a polymorphism manifested as occurrence of a codon encoding either threonine or methionine at codon 174 of the open reading frame encoding angiotensinogen;
XVI) a polymorphism manifested as occurrence of either a tetra-guanine repeat or a penta-guanine repeat near nucleotide residue −675 of the promoter operably linked with the open reading frame encoding plasminogen activator inhibitor 1;
XVII) a polymorphism manifested as occurrence of a codon encoding either arginine or glutamine at codon 506 of the open reading frame encoding coagulation factor V;
XVIII) a polymorphism manifested as occurrence of a codon encoding either glycine or tryptophan at codon 460 of the open reading frame encoding alpha adducin I;
XIX) a polymorphism manifested as occurrence of either a cytosine residue or a thymine residue at nucleotide residue −344 of the promoter operably linked with the open reading frame encoding cytochrome P450;
XX) a polymorphism manifested as occurrence of either a cytosine residue or a thymine residue at nucleotide residue 825 of the open reading frame encoding G protein beta, polypeptide 3;
XXI) a polymorphism manifested as occurrence of a codon encoding either isoleucine or methionine at codon 22 of the open reading frame encoding methionine synthase reductase;
XXII) a polymorphism manifested as occurrence of a codon encoding either serine or arginine at codon 128 of the open reading frame encoding endothelial adhesion molecule 1;
XXIII) a polymorphism manifested as occurrence of either a cytosine residue or an adenosine residue at nucleotide residue −629 of the promoter operably linked with the open reading frame encoding cholesteryl ester transferase; and
XXIV) a polymorphism manifested as occurrence of either a guanine residue or an adenosine residue at nucleotide residue −971 of the promoter operably linked with the open reading frame encoding cholesteryl ester transferase.

65. A method of assessing the advisability that a human should employ a nutritional product comprising a cardiovascular protective agent, the method comprising assessing occurrence in the human's genome of disorder-associated polymorphisms in at least two genes selected from the group consisting of
a) the gene which encodes apolipoprotein B;
b) the gene which encodes apolipoprotein E;
c) the gene which encodes paraoxonase 1;
d) the gene which encodes type 1 angiotensin II receptor;
e) the gene which encodes cytochrome b-245(alpha);
f) the gene which encodes prothrombin;
g) the gene which encodes coagulation factor VII;
h) the gene which encodes platelet glycoprotein 1b alpha;
i) the gene which encodes platelet glycoprotein IIIa;
j) the gene which encodes endothelial nitric oxide synthase;
k) the gene which encodes 5,10-methylene tetrahydrofolate reductase;
l) the gene which encodes angiotensinogen;
m) the gene which encodes plasminogen activator inhibitor 1;
n) the gene which encodes coagulation factor V;
o) the gene which encodes alpha adducin I;
p) the gene which encodes cytochrome P450;
q) the gene which encodes G protein beta, polypeptide 3;
r) the gene which encodes methionine synthase reductase;
s) the gene which encodes endothelial adhesion molecule 1; and
t) the gene which encodes cholesteryl ester transferase,
whereby occurrence of any of the polymorphisms is an indication that it is more advisable for the human to employ the nutritional product than a human whose genome does not comprise the polymorphism, and whereby occurrence of a plurality of the polymorphisms is an indication that it is even more advisable that the human should employ the nutritional product than a human whose genome does not comprise the polymorphisms.

66. A method of selecting a dose of a cardiovascular protective agent for administration to a human in a nutritional product, the method comprising assessing occurrence in the human's genome of disorder-associated polymorphisms in at least two genes selected from the group consisting of
a) the gene which encodes apolipoprotein B;
b) the gene which encodes apolipoprotein E;
c) the gene which encodes paraoxonase 1;
d) the gene which encodes type 1 angiotensin II receptor;
e) the gene which encodes cytochrome b-245(alpha);
f) the gene which encodes prothrombin;
g) the gene which encodes coagulation factor VII;
h) the gene which encodes platelet glycoprotein 1b alpha;
i) the gene which encodes platelet glycoprotein IIIa;
j) the gene which encodes endothelial nitric oxide synthase;
k) the gene which encodes 5,10-methylene tetrahydrofolate reductase;
l) the gene which encodes angiotensinogen;
m) the gene which encodes plasminogen activator inhibitor 1;
n) the gene which encodes coagulation factor V;
o) the gene which encodes alpha adducin I;
p) the gene which encodes cytochrome P450;
q) the gene which encodes G protein beta, polypeptide 3;
r) the gene which encodes methionine synthase reductase;
s) the gene which encodes endothelial adhesion molecule 1; and
t) the gene which encodes cholesteryl ester transferase,
whereby occurrence of any of the polymorphisms is an indication that a greater dose of the agent should be administered to the human in the nutritional product; and
selecting a dose of the agent for the nutritional product based on occurrence of the polymorphisms.
